(19) United States
(12) Patent Application Publication
(10) Pub. No.: US 2024/0018591 A1
(43) Pub. Date: Jan. 18, 2024

(54) A GENE ASSOCIATED WITH HUMAN READING PERFORMANCE

(71) Applicants: Yale University, New Haven, CT (US); Brock University, St. Catherines (CA)

(72) Inventors: Jeffrey R. Gruen, Hamden, CT (US); Andrew Adams, New Haven, CT (US); Dongnhu Truong, Rocky Hill, CT (US); Jan Frijters, Pelham (CA); Joan Bosson-Heenan, New Haven, CT (US)

(73) Assignees: Yale University, New Haven, CT (US); Brock University, St. Catherines, ON (CA)

(21) Appl. No.: 17/766,957

(22) PCT Filed: Oct. 8, 2020
(86) PCT No.: PCT/US2020/054790

§ 371 Date: Apr. 6, 2022

Related U.S. Application Data
(60) Provisional application No. 62/912,625, filed on Oct. 8, 2019.
(60) Provisional application No. 62/915,594, filed on Oct. 15, 2019.

(51) Int. Cl. C12Q 1/6883

(52) U.S. Cl. CPC C12Q 1/6883; C12Q 2600/156

(57) ABSTRACT

Disclosed herein are kits and methods for assessing the risk of poor reading performance in an individual by detecting and identifying single nucleotide polymorphisms in chromosome 19, e.g. in the KIAA0355 (GARRE1) gene. Also disclosed herein are risk alleles in chromosome 19 that are associated with a latent measure for reading ability.

### RELATED APPLICATIONS

This application is a national stage filing under 35 U.S.C. § 371 of international application number PCT/US2020/054790, filed Oct. 8, 2020, which claims the benefit of priority under 35 U.S.C. § 119(e) of U.S. Provisional Application Ser. No. 62/912,625, filed on Oct. 8, 2019 and U.S. Provisional Application Ser. No. 62/915,594, filed on Oct. 15, 2019. Each of the referenced applications is incorporated herein in its entirety by reference.

### GOVERNMENT SUPPORT

This invention was made with government support under HD027802 awarded by the National Institutes of Health. The government has certain rights in the invention.

### BACKGROUND

Specific learning disabilities (LDs) are disorders characterized by unexpected difficulty with a specific mode of learning, despite adequate IQ and educational opportunity. LDs can involve reading, math, writing, and speech skills, among others, but the most common involve language. It is estimated that about 3-10% of people have specific difficulties in reading, despite adequate intelligence, education and social environment. The National Institute of Child Health and Development (NICHD) estimates 15-20% of Americans have a language-based LD, of which reading disability (RD) afflicts the majority. Examples of reading disabilities include: developmental dyslexia, alexia (acquired dyslexia), and hyperlexia (word-reading ability well above normal for age and IQ).

### SUMMARY

The present disclosure relates, at least in part, to methods and kits for analyzing human nucleic acid for one or more nucleotides in human chromosome 19 that show an association with a latent measure of reading ability.

One aspect of the present disclosure provides a method of analyzing human chromosome 19 comprising detecting, in a human sample obtained from an individual and comprising nucleic acid, the identity of at least one single non-coding single nucleotide polymorphism (SNP) that has a reference sequence (rs) number listed in Table 2 or a reference sequence (rs) number listed in Table 3, or a SNP listed in Table 6, or an rs number listed in Table 6, wherein the nucleotide identity of the at least one single nucleotide polymorphism (SNP) is the corresponding risk allele listed in Table 2 or Table 6.

Another aspect of the present disclosure provides a method of detecting one or more single nucleotide polymorphisms (SNPs) in human chromosome 19 in a sample, wherein the SNPs have any one of the reference sequence (rs) numbers listed in Table 2 or any one of the reference sequence (rs) numbers listed in Table 3, or any one of the SNPs listed in Table 6, or any one of the rs numbers listed in Table 6, wherein the identity of the SNPs determines (is associated with, or indicative of) the risk of poor reading performance in an individual, and wherein the sample is obtained from an individual and comprises nucleic acid.

In some embodiments, the presence of a minor allele at any one of the SNPs indicates the presence or predisposition for poor reading performance.

Another aspect of the present disclosure provides a method of assessing the risk of low reading performance in an individual, the method comprising detecting, in a sample obtained from an individual, the identity of at least one single nucleotide polymorphism (SNP) having a reference sequence number listed in Table 2 or a reference sequence number listed in Table 3, or an SNP listed in Table 6, or an rs number listed in Table 6, wherein the nucleotide identity of the at least one SNP is the corresponding risk allele according to Table 2 or Table 6, wherein the sample comprises nucleic acid.

Another aspect of the present disclosure provides a method of detecting the presence of, or predisposition for, low reading performance in an individual, comprising detecting, in a sample obtained from the individual, the identity of at least one single nucleotide polymorphism (SNP) having a reference sequence number listed in Table 2 or a reference sequence number listed in Table 3, or a SNP listed in Table 6, or an rs number listed in Table 6, wherein the nucleotide identity of the at least one single nucleotide polymorphism is the corresponding risk allele according to Table 2 or Table 6, wherein the sample comprises nucleic acid.

Another aspect of the present disclosure provides a method of assessing the risk of low reading performance, the method comprising detecting the identity of at least one single nucleotide polymorphism (SNP) in the KIAA0355 gene on chromosome 19 (19q13.11), wherein the identity of the SNP is associated with a latent measure of reading ability.

In some embodiments, the detecting is performed in a sample obtained from an individual, wherein the sample comprises nucleic acid. In some embodiments, the SNP is a non-coding SNP. In some embodiments, the latent measure of reading ability is decoding ability.

In some embodiments, the SNP has any one of the reference sequence (rs) numbers listed in Table 2 or Table 6 or any one of the reference sequence (rs) numbers listed in Table 3 or Table 6 and is located within base pair locations (BP) 34,348,356-34,359,412, wherein the presence of a minor allele at any one of the reference sequence numbers indicates the presence of or predisposition for poor reading ability.

Another aspect of the present disclosure provides a method of assessing the risk of low reading performance in an individual, comprising detecting the identity of at least one single nucleotide polymorphism (SNP) having a reference sequence (rs) number listed in Table 2 or a reference sequence (rs) number listed in Table 3, or a SNP listed in Table 6, or an rs number listed in Table 6, wherein the nucleotide identity of the at least one single nucleotide polymorphism is the corresponding risk allele according to Table 2 or Table 6.

In some embodiments, the detecting is performed in a sample obtained from an individual, wherein the sample comprises nucleic acid. In some embodiments, the SNP has a reference sequence number of rs1669263 and a nucleotide identity of C. In some embodiments, the SNP has a reference sequence number of rs2599553 and a nucleotide identity of A. As described herein, SNPs, such as rs1669623 and the corresponding nucleotide identity C, and SNP rs2599553 and the corresponding nucleotide identity A, are identified in (or, identify) individuals whose reading performance, as assessed using reading measures (e.g., those described herein) is not as strong as the performance of individuals who do not have the SNP and corresponding nucleotide identity, such as SNP rs1669623 and nucleotide C or SNP rs2599553 and nucleotide identity A. For example, if the individual has the SNP having a reference sequence number of rs2599553 and a nucleotide identity of A, the individual's reading performance, as assessed by appropriate reading measures, is lower than an individual who does not have the SNP having a reference sequence number of rs2599553 and a nucleotide identity of A. In some embodiments, the reading performance is measured by at least one of: letter word identification, word attack, passage comprehension, and reading fluency.

In some embodiments, the detecting comprises nucleic acid sequencing techniques. In some embodiments, the detecting comprises using next generation sequencing or microarray genotyping. In some embodiments, the sample is saliva, blood, or urine. In some embodiments, the sample is saliva. In some embodiments, the SNP is on human chromosome 19 and in KIAA0355 (GARRE1), GPI, PDCD2L, or UBA2. In some embodiments, the SNP is non-coding.

In some embodiments, the individual has any one of the risk alleles in Table 2 or Table 3 or Table 6, the method further comprises monitoring the individual from whom the sample was obtained to assess whether development of a learning or reading disability occurs and if development occurs, treating the individual for the learning or reading disability, wherein treating comprises providing interventions, including services and materials, including but not limited to: using special teaching techniques; making classroom modifications, such as providing extra time to complete tasks and taped tests to permit the individual to hear, rather than read, the tests; using books on tape; using word-processing programs with spell-check features; helping the individual learn through multisensory experiences; teaching coping tools; and providing services to strengthen the individual's ability to recognize and pronounce words.

In some embodiments, if the individual has any one of the risk alleles in Table 2 or Table 6, the method further comprises administering an intelligence quotient (IQ) test.

Another aspect of the present disclosure provides a method of analyzing human chromosome 19 (19q13.11) by detecting in a sample, obtained from a human and comprising nucleic acid, at least one non-coding single nucleotide polymorphism (SNP) having a reference sequence (rs) number in Table 2 or a reference sequence (rs) number in Table 3, or a SNP listed in Table 6, or an rs number listed in Table 6, comprising:
(a) combining the sample with polynucleotides that hybridize, under highly stringent conditions, with the at least one non-coding SNP when the nucleotide identity of the at least one-non SNP corresponds is the corresponding risk allele in Table 2 or Table 6; and
(b) determining whether hybridization of the polynucleotides in (a) occurs, wherein the occurrence of hybridization the polynucleotides indicates that the human has the risk allele and is susceptible to or has developed a reading disability.

In some embodiments, a reading disability is also referred to as “low reading performance”, “poor reading performance”, “low reading ability”, or “poor reading ability”.

In some embodiments, one SNP from Tables 2, 3, or 6 is detected. In some embodiments, a subset of the SNPs from Tables 2, 3, and/or 6 is detected. In some embodiments, all the SNPs in Table 2 or Table 3 or Table 6 are detected. Any combination of SNPs from Table 2 or Table 3 or Table 6 may be detected in the present method.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure. In the drawings:

FIG. 1 includes a flow chart showing the methods used in the logistic regression based genome wide association study (GWAS) to identify genetic variants that are associated with performance on a latent measure of reading ability.

FIG. 2 includes a histogram of readingT3nocovar.

FIG. 3 includes a histogram of readingT3nocovar divided into case/control status based on performance above or below the mean.

FIG. 4 includes a Manhattan plot for Primary GWAS (n=359). Using a case-control dichotomization of readingT3nocovar, a peak of SNPs was identified on chromosome 19. With a lambda value of 1.03, these results seem not to be inflated by cryptic ancestry. The peak represents the 39 top SNPs described in Table 2.

FIG. 5 includes a diagram showing a UCSC Genome Browser view of top SNPs overlapping with genes. rs1669263 is highlighted with a vertical line and is the SNP that had the lowest p-value in Table 2 (also referred to as “top SNP”).

FIG. 6 includes a diagram showing eQTL data from the GTEx project for the top SNP rs1669263 and KIAA0355 across all sampled brain regions. Of all brain regions, rs1669263 was shown to be a significant eQTL for expression in the cerebellum, with homozygous reference individuals having increased expression of KIAA0355.

FIG. 7 includes a plot showing GTEx gene expression data for KIAA0355. Peak median expression of >20 transcripts per million were seen in the cerebellar hemispheres.

FIG. 8 includes a plot showing quantile regression for rs1669263. The risk allele of rs1669263 has differing effects across different quantiles of the latent reading variable spectrum, with significant effects only observed between the 25th and 75th percentiles of the raw variable. A recent study by Pozarickij et al. (Communications Biology 2, 1-8 (2019)) suggests that these results may be due to either GxE or GxG interactions among subjects in the tails. Reading and language development are expected to have significant GxE interactions in particular, especially around SES, though more work is needed to untangle this in the New Haven Lexinome Project (NHLP).

FIG. 9 includes a plot showing the genetic variants (single nucleotide polymorphisms) associated with performance on the latent measure for reading ability along with their recombination rates, positions on chromosome 19 and positions relative to certain genes on chromosome 19. The plot is a zoomed regional plot of the segment of chromosome 19 (chr19:34742162-chr19:34943280, reference genome: hg19). The single peak of association at chr19:34816031 shows p-value=1.014×10⁻⁹.

FIG. 10 includes a histogram of readingT3nocovar divided into case/control status based on performance above or below the mean.

FIG. 11 is a plot of PC1 versus PC2 for the sequenced NHLP merged with 1000 genomes. Plotting PC1 vs. PC2 shows population structure in the NHLP. PCA indicates that NHLP subjects are predominantly Hispanic or African American with small numbers of Europeans and Asians. AFR=African Superpopulation; AMR=Admixed American Superpopulation; EAS=East Asian Superpopulation; EUR=European Superpopulation.

FIG. 12A shows a Manhattan plot of primary GWAS. The upper line indicates the genome wide significance threshold of 5×10⁻⁸. The lower line indicates a suggestive threshold of 1×10⁵. P-values are negative log transformed.

FIG. 12B is a Q-Q plot corresponding to GWAS results in FIG. 12A.

FIG. 13 shows the raw decoding composite scores for each self-report racial grouping (N=415). DUAL indicates more than one category selected; MISS indicates no data at this question.

FIGS. 14A-14D relate to ancestry-specific GWAS for chromosome 19 locus, highlighting lead SNP rs2599553 (labeled). The shade of SNPs corresponds to the level of correlation with rs2599553 using the 1000 Genomes EUR LD map. SNP location and density are visualized at the top of each image. Genes in the area are visualized in the bottom track. FIG. 14A shows the results in the African (AFR) population, FIG. 14B shows the results in the Admixed American (AMR) population, FIG. 14C shows the results for the European (EUR) population, and FIG. 14D presents the summary statistics from the individual GWAS and the meta-analysis.

FIGS. 15A-15B show the BrainSpan expression of GARRE1. FIG. 15A shows expression of GARRE1 for all fetal samples (12-37pcw, N=11). FIG. 15B shows expression of GARRE1 for post-natal samples (4 months-40 years, N=21). Expression data suggest relatively constant expression of GARRE1 in the cerebellum, while expression in the rest of the brain drops.

FIG. 16 shows a marginal slopes plot for minor alleles of rs2599553 and decoding performance. The lower line represents the slope of the regression for the bottom 25% of subjects in the GRaD by age, the middle line represents the slope of the regression for the center 50% of subjects in the GRaD by age, and the top line represents the slope of the regression for the top 25% of subjects by age.

FIG. 17A-17B show growth curves relating to children's performance relative to developmental expectations with and without the rs2599553 minor allele. The raw data is given in FIG. 17A and the standard scores are shown in FIG. 17B.

Tables

Table 1 is a list of measures included in the New Haven Lexinome Project (NHLP). Table 2 lists SNPs associated with reading performance. The nucleotide in the risk allele column is the nucleotide that showed association with the reading phenotype at the corresponding P-value depicted in Column P. The SNPs in Table 2 are all non-coding—they do not change an amino acid in a protein. None of the nucleotides listed in the risk allele column correspond with the reference allele at the base pair location listed in the base pair location column. They are the minor alleles (defined as occurring at a lower frequency than the major alleles) for each SNP within the New Haven Lexinome Project (NHLP) sample. The location of the SNP in the “base pair location” column is as assigned in reference genome hg19 (also known as Genome Reference Consortium Human Build 37 (GRCh37) as described at ncbi.nlm.nih.gov/assembly/GCF_000001405.13/). None of the SNPs change an amino acid in any protein and are referred to as risk alleles. The nucleotides in the risk allele column indicate an increased risk of low reading performance. The reported p-values are for association between the risk allele and the phenotype, which here is performance on a latent measure of reading ability in grade school children, after controlling for ancestry, socioeconomic status, age, and sex.

Table 3 is a list of SNPs associated with reading performance as identified in replicated studies using a separate sample (n=703) of age-matched children drawn from the Genes Reading and Dyslexia (GRaD) study. Of the 39 top SNPs from NHLP, 32 were present in the imputed GRaD dataset.

Table 4: NHLP GWAS sample demographics (N=407 with all covariate data) (see Example 2).

Table 5: Measures performed in the NHLP (measures included in the decoding composite phenotype are in bold).

Table 6: Primary GWAS results for chromosome 19 sorted by base pair position. Significant or suggestive SNPs are reported. BP is in hg19 coordinates. Minor is the minor allele and OR is odds ratio from PLINK. Top SNP, rs2599553, is highlighted.

Table 7: Chi-squared test for difference of minor/major allele counts across self-report identities.

Table 8: One-way ANOVA results for differences in mean across self-report racial groupings.

Table 9: GRaD candidate SNP replication. Columns are SNP ID, number of subjects included in the model, and P-value from logistic regression.

Table 10: GRaD moderation analysis results. In the model summarized in the left column, there is no interaction term. Age is not a significant predictor of decoding performance. In the model summarized in the right column, the SNP by decoding relationship is moderated by age. Both the SNP and the moderation term are significant, suggesting age moderates the relationship between rs2599553 and decoding.

Table 11: Woodcock-Johnson III Raw Score Mean Differences (Example 2).

Table 12: Woodcock-Johnson III Standard Score Mean Differences (Example 2).

Table 13: Random Effects Covariance Parameter Estimates for Woodcock-Johnson III Raw Scores (Example 2).

Table 14: Random Effects Covariance Parameter Estimates for Woodcock-Johnson III Standard Scores (Example 2).

### DETAILED DESCRIPTION

Described here are methods and kits for analyzing human nucleic acid (e.g., chromosomal DNA; mRNA) for one or more nucleotides in human chromosome 19, such as one or more of the nucleotides shown in Table 2 or Table 6, risk allele column, that show an association with a latent measure of reading ability. Identifying genetic variants or markers that can be used to predict or detect reading disabilities is important in optimizing intervention strategies for individuals with reading disability. As described, a human gene (e.g., the human gene, KIAA0355, also referred to herein as “GARRE1”) has unexpectedly been shown to be associated with reading performance on a latent measure of reading ability in grade school children, after controlling for multiple factors, such as but not limited to, ethnicity, ancestry, socioeconomic status, age, and sex. The present disclosure provides genetic variants (e.g. risk alleles) that are found in human chromosome 19 and are correlated, at a high statistical significance, with poor reading performance. In some embodiments, these genetic variants exceeded the standard threshold for genome wide statistical significance (p-value<5×10⁻⁸).

Provided herein are methods for analyzing human chromosome 19, comprising detecting the identity of one or more single nucleotide polymorphisms (SNPs). If the one or more SNPs are genetic variants (also referred to herein as “risk alleles”), then that is indicative of the presence of a reading disability or the predisposition for a reading disability in a human, such as a school-aged child.

As used, the terms “genetic variant” are “risk allele” are used interchangeably. The term “genetic variant” refers to an alteration in the most common nucleotide sequence. Generally, genetic variants can be benign, pathogenic, or have an unknown role. The present disclosure relates to genetic variations that are associated with poor reading performance (see, for example, the SNPs in Table 2 or Table 6, which are all non-coding) and can be used to identify an individual having a reading disability. The term “risk allele” refers to the nucleotide identity of one of these SNPs and is the nucleotide identity that indicates the susceptibility or the presence of a reading disability in an individual. The minor allele (also referred to as the less common allele) at each of the SNP locations is associated with a reading disability.

The majority of genetic variants in the present disclosure are SNPs located on a single gene, referred to here as human gene KIAA0355 (GARRE1). This, combined with the fact that the genetic variants disclosed herein are each correlated with reading disability at a highly statistically significant level (e.g., p-value<5×10⁻⁸), makes it possible to assess reading performance in young children and provide intervention for those children identified as having or likely to develop low reading performance. The methods disclosed make it possible to rely on screening of a small number of SNPs (in some embodiments, a single SNP can be used to screen), which enables rapid screening at low cost. This improves accessibility to screening for various demographics (e.g. various socioeconomic groups). The methods disclosed herein make it possible to assess the risk of learning disability in young children (e.g., grade school children) and allow for early intervention measures that, in turn, give individuals greater access to educational and occupational opportunities.

Reading Disability

Developmental reading disabilities have been classified into three groups, which can overlap in individuals or manifest as separate and distinct disabilities. The three groups are: (i) phonological deficit, which is a problem or failure in the phonological processing system of oral language; (ii) processing speed/orthographic processing deficit (also referred to as naming speed problem or a fluency problem), which affects the speed and accuracy of printed word recognition; and (iii) comprehension deficit, which commonly occurs in individuals having social-linguistic disabilities (e.g., autism spectrum), vocabulary weaknesses, generalized language learning disorders, and learning difficulties that affect abstract reasoning and logical thinking.

Alternatively, reading disability can be classified into three types: (i) inability to decode, (ii) inability to comprehend or (iii) both (Gough, Philip B., and William E. Tunmer. Remedial and special education 7.1 (1986): 6-10).

Intelligence Quotient (IQ) Testing

Traditionally, reading disability was treated as a disorder that manifests as a discrepancy in intellectual aptitude and adequate opportunity to learn. Children were administered intelligence quotients (IQ) tests and reading disability was diagnosed based on the difference between IQ scores and scores on a test of reading achievement. The specific discrepancy required for a diagnosis varied from one state to another and would determine whether children were granted access to special education services under the “learning disabilities” label. This meant that some children who were susceptible to developing more severe reading problems with time were deprived of intervention. A small number of schools would qualify students as having learning disabilities based on professional judgment rather than IQ-achievement discrepancies.

In some cases, there is a heavy reliance on IQ-achievement discrepancy, which precludes a subset of children having reading disabilities from receiving adequate intervention. The methods and kits of the present invention may allow the identification of a greater number of individuals who are susceptible to developing reading disability and allow for earlier intervention.

In some embodiments of the present disclosure, the methods of the present disclosure are combined with IQ testing of an individual, such as a grade school-aged child. In some embodiments, IQ testing is performed prior to, concurrently with, or after performing the methods of the present disclosure.

In some embodiments, if an individual has an IQ-achievement discrepancy that would qualify the individual as having (is indicative of their having) a reading disability and the individual has a genetic variant associated with susceptibility to/increased likelihood of developing a reading disability or associated with the presence of a reading disability, as disclosed herein, the individual, such as a grade school-aged child, is given access to/should be provided with appropriate intervention measures.

In some embodiments, if an individual (e.g., a grade school-aged child) does not have an IQ-achievement discrepancy that would qualify the individual as having (is indicative of their having) a reading disability but the individual has a genetic variant associated with susceptibility to/increased likelihood of developing a reading disability or associated with the presence of a reading disability, as disclosed herein, the individual, such as a grade school-aged child, is given access to/should be provided with appropriate intervention measures.

Measures for Reading Ability

The present invention relates to methods and kits for detecting genetic variants that indicate susceptibility or the presence of reading disability in an individual (e.g., a grade school-aged child). These genetic variants are disclosed in Tables 2 and 6 and most of them are located on human gene KIAA0355 (falling within the 34,348,356-34,359,412 base pair location on chromosome 19), thus showing the first association of KIAA0355 with reading performance. The genetic variants disclosed herein were identified based on performance on a latent measure of reading ability created with measurements of decoding related tasks. The decoding related tasks were used to create the latent measure referred to herein as the “readingT3nocovar variable”.

The present disclosure teaches that statistically significant association between any of the genetic variants (e.g. p<0.05, p<0.01, p<0.001, p-value<5×10⁻⁸) and the latent measure, readingT3nocovar, is indicative of impairment in decoding related tasks and susceptibility to, if not presence of, a reading disability in an individual, such as grade school or grade school-aged children.

A “latent measure” is a variable that is not directly observed but inferred (e.g., through mathematical modeling) from other variables. The latent measure in the present invention is reading T3nocovar, which was created using decoding related tasks. An example of a decoding related task is having an individual view a combination of letters (e.g., presented as a single word) and identify whether the combination of letters is an actual word or a random combination of letters that does not qualify as a word. This decoding related task controls for languages (e.g., the actual words are in the language of the individual).

Non-limiting examples of reading measures are shown in Table 1 below.

TABLE 1
Reading measures included in the New Haven Lexinome Project (NHLP).
Measure

Peabody Picture Vocabulary Test, 4th Ed. (PPVT-4)
Test of Word Reading Efficiency, Second Ed. (TOWRE−2)
Woodcock-Johnson III Tests of Achievement (WJ III ACH)
Comprehensive Test of Phonological Processing, Second Ed. (CTOPP-2)
Gray Oral Reading Tests, Fifth Ed. (GORT-5)
Clinical Evaluation of Language Fundamentals Screening Test, Fifth Ed.
(CELF-5 Screening Test)
Wechsler Abbreviated Scale of Intelligence, Second Ed. (WASI-II Full)
A Developmental Neuropsychological Assessment, Second Ed.
(NEPSY-II)
Wechsler Intelligence Scale for Children, Fourth Ed. (WISC-IV)
Barkley ADHD Screening Checklist Rating Scale
Strengths & Weaknesses in ADHD-Symptoms & Normal Behavior
(SWAN)

Interventions

Generally, intervention is more effective the earlier it is provided, which underscores the importance of early detection of high-risk individuals. Several research studies have demonstrated that it is most effective in primary grades (e.g., elementary grades) of school and children of similar age (early school-aged children) and it effectively reduces the severity of the reading problems as the children age.

A 2001 analysis of response rates to interventions estimated that the number of students experiencing serious reading problems could be reduced from about 20% to 5% or less of the school population through quality early intervention. (See Lyon, G. R. et al. 2001. In Rethinking Special Education for a New Century, ed. Chester E. Finn, Andrew J. Rotherham, and Charles R Hokanson, Jr. Washington, DC: Fordham Foundation, the relevant disclosures of which are herein incorporated by reference for the purpose and subject matter referenced herein).

In some embodiments of the present disclosure, the interventions include, without limitation, monitoring the individual from whom the sample was obtained to assess whether development of a learning or reading disability occurs and if development occurs, treating the individual for the learning or reading disability, wherein treating comprises providing interventions, including services and materials, including but not limited to: using special teaching techniques; making classroom modifications, such as providing extra time to complete tasks and taped tests to permit the individual to hear, rather than read the tests; using books on tape; using word-processing programs with spell-check features; helping the individual learn through multisensory experiences; teaching coping tools; and providing services to strengthen the individual's ability to recognize and pronounce words.

Single Nucleotide Polymorphisms

A single-nucleotide polymorphism (SNP) is a substitution of a single nucleotide that occurs at a specific position in the genome. It occurs when a single nucleotide varies between members of a species or paired chromosome in an individual. The possible nucleotide variations at that specific position are referred to as alleles for the position. The “major allele” is present at a higher frequency than the minor allele(s). It is possible to have more than one minor allele.

The SNPs in in Tables 2 and 6 can be used for assessing risk of reading problems in children with different ancestral backgrounds: Hispanic American, African American, and European descent, for example. The number of SNPs is small, and they implicate a single gene. As a result, large-scale screening for risk of reading difficulties could be deployed at low cost.

The genetic variants disclosed herein are non-coding SNPs, which means that they do not encode or change an amino acid.

SNP Detection Methods

In some embodiments, the SNPs of the present disclosure are detected using allele-specific probes. Allele specific probes are known in the art and are designed to hybridize to complementary target sequences only when there is, for example 100%, complementarity between the probe and the target sequence. Under optimized or stringent conditions, a single-base mismatch can prevent the annealing of an allelic probe to a sequence.

Complementary, as the term is used in the art, refers to the capacity for precise pairing between two nucleotides. For example, if a nucleotide at a certain position of an oligonucleotide is capable of hydrogen bonding with a nucleotide at a corresponding position of a target nucleic acid, then the nucleotide of the oligonucleotide and the nucleotide of the target nucleic acid are complementary to each other at that position. The oligonucleotide and target nucleic acid are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and target nucleic acid sequence. For example, if a base at one position of an oligonucleotide is capable of hydrogen bonding with a base at the corresponding position of a target, then the bases are considered to be complementary to each other at that position. 100% complementarity is not required.

An oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a target. In some embodiments an oligonucleotide may contain 1, 2 or 3 base mismatches compared to the portion of the consecutive nucleotides of the target. In some embodiments an oligonucleotide may have up to 3 mismatches over 15 bases, or up to 2 mismatches over 10 bases.

In some embodiments, allelic probes can be immobilized on a solid support and target DNA samples hybridize to the immobilized probes. The unbound DNA is removed with a rinsing step and the genotype of the SNP can be inferred from the locations of hybridization on the solid support.

In some embodiments, the probes fluoresce to indicate hybridization to a target sequence and allow identification of a SNP of interest.

In some embodiments, the SNPs of the present disclosure are detected using a DNA microarray or a SNP array. In some embodiments, the use of a microarray comprises the use of allele-specific oligonucleotide probes, target sequences (e.g. fragmented nucleic acid sequences of the target), fluorescent dyes or fluorophores for labeling the target sequences. In some embodiments, at least two probes are used per SNP to detect the major and minor allele. In some embodiments, the number of SNPs is the same as the number of alleles at the SNP of interest.

Other methods for genotyping SNPs include, without limitation, primer extension, ligation (e.g. use of DNA ligase to identify SNPs), invasive cleavage, reactions formats, homogeneous reactions, reactions on solid supports, detection mechanisms (e.g. based on light emission, mass of products, change in electrical properties of products, etc.), luminescence detection, fluorescence detection, fluorescence resonance energy transfer (FRET), fluorescence polarization (FP), mass spectrometry, and electrical detection. Methods for genotyping SNPs are provided in Kwok, Pui-Yan, and Xiangning Chen. “Detection of single nucleotide polymorphisms.” (2003), the relevant disclosures of which are herein incorporated by reference for the purpose and subject matter referenced herein.

In some embodiments, the detection of a SNP of the present disclosure is performed using a technique selected from the group consisting of a padlock probe, the probe molecules reverse, other circular probe, genotypes microarray, SNP genotyping, microarray, bead microarrays, SNP microarrays other, other genotyping method, Sanger DNA sequencing, pyrosequencing, high-throughput sequencing, the use of probes directed annular sequencing, hybridization using capture probes directional sequencing, reversible dye terminator sequencing, sequencing by ligation, sequencing by hybridization other DNA sequencing, other high-throughput genotyping platforms, fluorescent in situ hybridization (FISH), t dagger than genomic hybridization (CGH), CGH column array, as well as multiplication and combinations thereof.

Probes

In certain aspects of the disclosure, the genetic variants are detected by combining a sample from an individual with a polynucleotide (e.g. isolated or recombinant) or probe that hybridizes to one or more of the genetic variants of the present disclosure (e.g., Tables 2 and 6). In some embodiments, this polynucleotide is a probe that hybridizes, under stringent conditions, such as highly stringent conditions, to a genetic variant that indicates susceptibility to reading disability, as described herein.

As used, the term “hybridization” refers to the pairing of complementary nucleic acids. The term “probe” refers to a polynucleotide that is capable of hybridizing to another nucleic acid of interest. The polynucleotide may be naturally occurring, as in a purified restriction digest, or it may be produced synthetically, recombinantly or by nucleic acid amplification (e.g., PCR amplification).

It is well known in the art how to perform hybridization experiments with nucleic acid molecules. The skilled artisan is familiar with hybridization conditions and that appropriate stringency conditions which promote DNA hybridization can be varied. Such hybridization conditions are referred to in standard textbooks such as Molecular Cloning: A Laboratory Manual, Cold Spring Harbor Laboratory (1989); and Current Protocols in Molecular Biology, eds. Ausubel et al., John Wiley & Sons: 1992.

A polynucleotide probe or primer used in a method described herein may be labeled with a reporter molecule, so that it is detectable in a detection system, including, but not limited to, enzyme (e.g., ELISA, as well as enzyme-based histochemical assays), fluorescent, radioactive, chemical, and luminescent systems. A polynucleotide probe or primer used in a method described herein may further include a quencher moiety that, when placed very close to a label (e.g., a fluorescent label), causes there to be little or no signal from the label. It is not intended that the present invention be limited to any particular detection system or label.

Nucleic acid hybridization is affected by such conditions as salt concentration, temperature, organic solvents, base composition, length of the complementary strands, and the number of nucleotide base mismatches between the hybridizing nucleic acids, as will readily be appreciated by those skilled in the art. Stringent temperature conditions will generally include temperatures in excess of 30° C., or may be in excess of 37° C. or 45° C. Stringent salt conditions will ordinarily be less than 1000 mM, or may be less than 500 mM or 200 mM. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6.0×SSC at room temperature followed by a wash at 2.0×SSC at room temperature. The combination of parameters; however, is much more important than the measure of any single parameter. See, e.g., Wetmur and Davidson, 1968. Probe sequences may also hybridize specifically to duplex DNA under certain conditions to form triplex or higher order DNA complexes. The preparation of such probes and suitable hybridization conditions are well known in the art. One method for obtaining DNA encoding the biosynthetic constructs disclosed herein is by assembly of synthetic oligonucleotides produced in a conventional, automated, oligonucleotide synthesizer.

Described herein is a method of analyzing human chromosome 19 (such as 19q13.11) by detecting, in a sample obtained from a human and comprising nucleic acid, at least one (a, one or more) non-coding single nucleotide polymorphism (SNP) having a reference sequence (rs) number that is listed and indicates the corresponding risk allele (referred to as a non-coding SNP listed)

SNP Ref Seq. (rs) No. Risk Allele
rs2115487 A
rs763199675 G
rs7359931 G
rs4805079 A
rs10426700 T
rs10407101 T
rs10407640 G
rs12975032 C
rs11671239 A
rs8110966 C
rs8105306 T
rs328412 A
rs328414 A
rs1664905 T
rs1669265 T
rs921476 A
rs1664904 T
rs2599553 A
rs1669263 C
rs2965269 A
rs189030 C
rs328400 G
rs1618249 T
rs328402 C
rs7254168 A
rs328406 T
rs328405 A
rs62122220 A
rs397072 A
rs385342 A
rs580391 T
rs422732 C
rs416602 C
rs452902 C
rs8191356 A
rs7260568 A
rs35024640 T
rs16969326 T
rs60857340 T
   comprising:
(a) combining a sample, obtained from a human and comprising nucleic acid, with polynucleotides that hybridize, under highly stringent conditions, with at least one of the non-coding SNPs listed and
(b) determining whether hybridization of the polynucleotides in (a) occurs, wherein the occurrence of hybridization polynucleotides indicates that a non-coding SNP listed and the corresponding risk allele are present in chromosome 19.

In some embodiments, the sample is combined with polynucleotides that hybridize (e.g., under highly stringent conditions) to at least two different non-coding SNPs listed (e.g., with polynucleotides that hybridize to rs2115487 and polynucleotides that hybridize to rs1669263). In further embodiments, the sample is combined with polynucleotides that each hybridize to two or more different non-coding SNPs listed (e.g., with polynucleotides that hybridize to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 different non-coding SNPs listed). In these embodiments, polynucleotides that hybridize to two or more different non-coding SNPs listed hybridize to one of the non-coding SNPs listed and not to more than one of the non-coding SNPs listed, in order to make it possible to distinguish between the non-coding SNPs listed and, thus, make it possible to determine whether the risk allele is in the sample.

In some embodiments, it is determined whether hybridization occurs and, if hybridization occurs, it is an indication that the human has the risk allele and is susceptible to or has a reading disability.

In a further embodiment, the non-coding SNP listed and the associated risk allele are those shown in Table 3 or Table 6.

Nucleic Acid Sequencing

In some embodiments, the sample from an individual (e.g., school-aged child) is analyzed by genetic sequencing (e.g. next generation sequencing). Amplified DNA is analyzed by DNA sequencing. DNA sequence determination may be performed by standard methods such as dideoxy chain termination technology (Sanger sequencing) and gel-electrophoresis, or by other methods such as by pyrosequencing (Biotage AB, Uppsala, Sweden).

Methods for nucleic acid sequencing are known to persons skilled in the art. Examples of nucleic acid sequencing methods include methods described in U.S. patent application publication numbers US 2006-0029957, US 2006-0024716, US 2006-0024717, US 2006-0024718 and US 2007-0134699, which are incorporated herein by reference. Other examples of sequencing include, without limitation, massively parallel signature sequencing (MPSS), polony sequencing, 454 pyrosequencing, Illumina (Solexa) sequencing, combinatorial probe anchor synthesis (cPAS), SOLiD sequencing, Ion Torrent semiconductor sequencing, DNA nanoball sequencing, Heliscope single molecule sequencing, single molecule real time (SMRT) sequencing, Sanger sequencing and nanopore DNA sequencing.

Analyzing Sequence Data

In some embodiments, the presence of a reading disability or susceptibility for a reading disability can be determined by analyzing a previously acquired sequence from an individual. A previously acquired sequence can be sequence data that was acquired in the past for purposes other than checking for a reading disability. The present disclosure provides methods for analyzing an individual's genome, comprising detecting in the sequence of an individual, the identity of at least one single nucleotide polymorphism (SNP) having a reference sequence number listed in Table 2 or a reference sequence number listed in Table 3, or a SNP listed in Table 6, or an rs number listed in Table 6, wherein the nucleotide identity of the at least one single nucleotide polymorphism is the corresponding risk allele according to Table 2 or Table 6, wherein the sample comprises nucleic acid.

Sample

Samples analyzed comprise nucleic acids, to allow for genotyping. A “sample” can be a body fluid sample, or a sample of cells isolated from body fluid, a tissue or organ sample. Non-limiting examples of body fluids include blood, blood matrix, serum, plasma, sputum, cerebrospinal fluid, breath condensate, saliva, urine, and tears. In some embodiments, the sample is saliva, blood, or urine. In some embodiments, the sample is blood, plasma or serum.

Methods of isolating body fluid samples are well known in the art and include, without limitation, blood drawing, venipuncture, finger-stick sampling, heel prick sampling, arterial blood sampling, lumbar puncture, paracentesis, thoracocentesis, amniocentesis, swabbing, and direct collection as the fluids exit the individual's body (e.g. an orifice).

Methods of isolating samples of cells or tissue are well known in the art and include, without limitation, swabbing, scraping, swiping, and biopsying.

In some embodiments of the present disclosure the detection of genetic variants is performed on cell free nucleic acids.

Individual

As used, the term individual refers to a human, particularly a child of school age, such as early school age (e.g., preschool, kindergarten, grade school, grades 1 through 6, grades 7 through 12 or the equivalent age), who can be of any gender or sexual identity.

The methods described are useful for assessing risk of reading problems in children of a variety of ancestral backgrounds. Non-limiting examples of ancestral backgrounds include Hispanic American, African American, and European descent. They are also useful in assessing risk of reading problems in a variety of races, including, but not limited to, American Indian or Alaska native, Asian, Black or African American, Native Hawaiian or other Pacific Islander, and white and a variety of ethnic categories, including, but not limited to, Hispanic or Latino and non-Hispanic or non-Latino.

Further, the method is applicable to assess the risk of reading problems in children from any socioeconomic status, which can be defined with reference to a variety of metrics, such as, but not limited to, highest level of education obtained by individual or household, education of parents or legal guardians, current occupation, and income.

In some embodiments, detection of one or more of the genetic variants of the present disclosure can be performed on an embryo (e.g., using embryo genotyping, e.g. by taking an embryo biopsy). In some embodiments, the detection of one or more of these genetic variants can be performed on a newborn, an infant, baby, toddler, pre-pubescent child, a child, a teenager, or an adult.

The detection of the genetic variants by any of the presently disclosed methods or by any method known in the art can be performed on an individual of any age.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Logistic Regression-Based Genome-Wide Association Study (GWAS) of 361 Subjects

The New Haven Lexinome Project (NHLP) is a longitudinal study of reading skill acquisition in children with normal and with atypical trajectories, and including intervention trials. The goal of the NHLP is to identify genetic variants associated with response-to-intervention that could be used at some future time to optimize intervention strategies for children with reading disability.

Using a logistic regression-based genome wide association study (GWAS) of 361 subjects (individuals) from New Haven Public Schools, it was shown that the human gene, KIAA0355, is associated with performance on a latent measure of reading ability in grade school children, after controlling for ancestry, socioeconomic status, age, and sex. This analysis identified 39 single nucleotide polymorphisms (SNPs) spanning 201,118 base pairs on chromosome 19. 33 of these SNPs exceeded the standard threshold for genome wide statistical significance (p-value<5×10⁻⁸). 28 SNPs are encoded within KIAA0355, supporting its association with reading performance. This represents the first reported association between KIAA0355 and reading performance.

### Introduction

Using a logistic regression based genome wide association study (GWAS) of 361 subjects drawn from the New Haven Public School District, it was demonstrated that the human gene, KIAA0355, is associated with performance on a latent measure of reading ability. This association was replicated using a separate sample of age-matched children drawn from the Genes Reading and Dyslexia (GRaD) study.

KIAA0355 (RefSeq: NM_014686) is a 101,016 base pair gene on chromosome 19 (19q13.11). It has base pair location 34,348,356-34,359,412. KIAA0355 has yet to be biochemically or functionally characterized, however, a previous large study of protein-protein interactions demonstrated an interaction between KIAA0355 and NCKAP1, an evolutionarily conserved gene involved in the cytoskeleton (Huttlin et al., 2017). Tissue specific RNA expression data support a neurological function with strong evidence for expression in human brain tissue from both the Genotype-Tissue Expression project (The GTEx Consortium, 2013) and the Brainspan project (Miller et al., 2014).

Methods

Recruitment for Wave 1 started in 2015 (374 enrolled). Wave 2 started in 2016. The entire Project is designed to continue through 2021. Following informed consent, children receive a comprehensive test battery with a concentration in the following domains: word reading/connected text, language, math, executive function, and reading-related cognition and motivation. Parents complete a questionnaire that asks about family history of learning difficulties, home life, language spoken in the home, and medical history of the child. A longitudinal sample and a treatment sample are being recruited from elementary schools in the New Haven public school district. Children in the longitudinal sample are being followed from Grade 1 through Grade 5, with multiple-measure assessments twice yearly. Children in the treatment sample (120 children total) were identified by having met school-based criteria for poor reading performance using end-of-Kindergarten risk scores on the Fountas and Pinnell Benchmark Assessment System. At-risk status is confirmed at the end of Grade 1. These children receive an intensive 100-hour reading intervention in each of Grades 2 and 3, and followed longitudinally through Grade 5. An at-risk sample of comparison children is being matched via propensity scores from schools not receiving the reading intervention. Based on participants to date, this sample is ethnically (e.g., 31% AA) and linguistically (e.g., 52.9% HA) diverse. DNA from each subject is being collected, extracted, and analyzed by whole genome sequencing. In addition, subjects are invited to participate in serial annual MRI studies beginning in Grade 2.

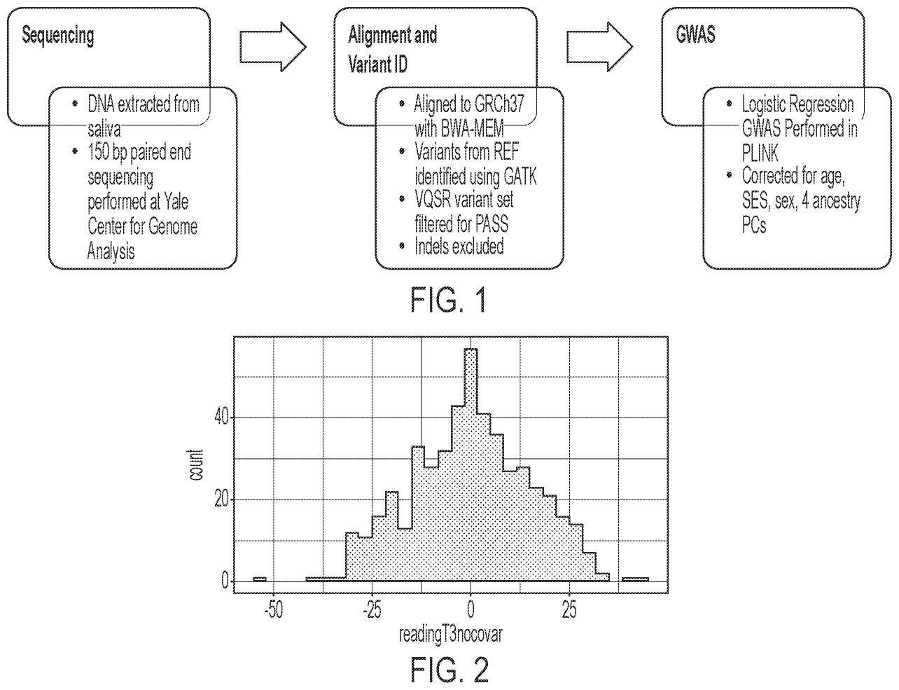

As illustrated in FIG. 1, the methods for the NHLP GWAS encompassed sequencing of samples from 361 subjects, alignment and genetic variant identification, and the regression analysis. First DNA was extracted from saliva samples and sequenced. Alignment was performed with Genome Reference Consortium Human Build 37 (GRCh37) with BWA-MEM, an algorithm in a software package for mapping low-divergent sequences against a large reference genome, such as the human genome. The variants were identified using the genome analysis toolkit (GATK). Then the Variant Quality Score Recalibrated (VQSR) variant set was filtered. Finally a logistic regression was performed in PLINK, which is a free, open-source whole genome association analysis toolset, designed to perform a range of basic, large-scale analyses in a computationally efficient manner.

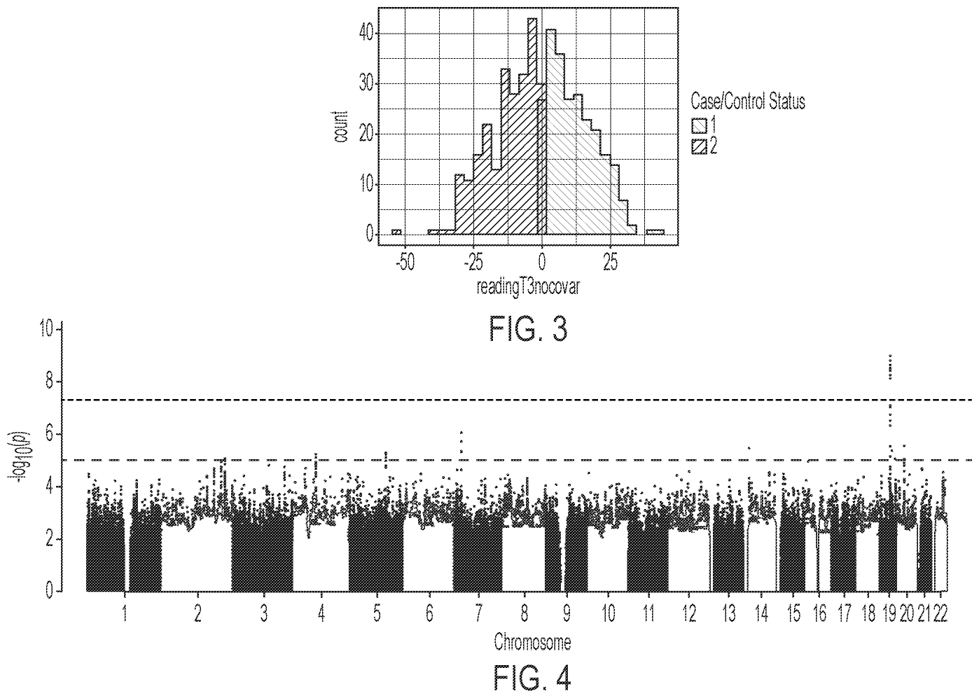

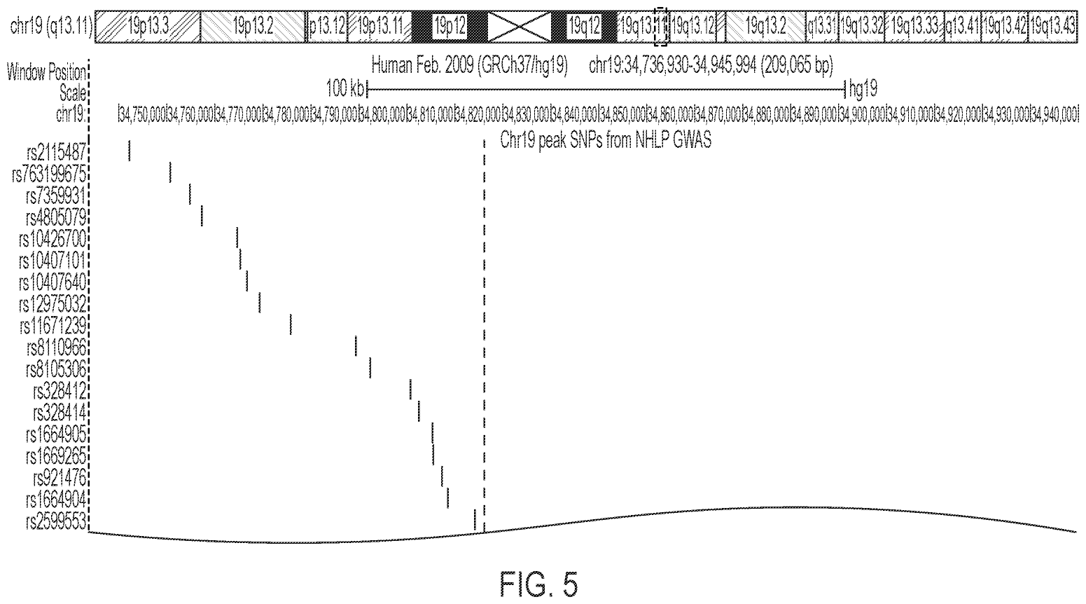

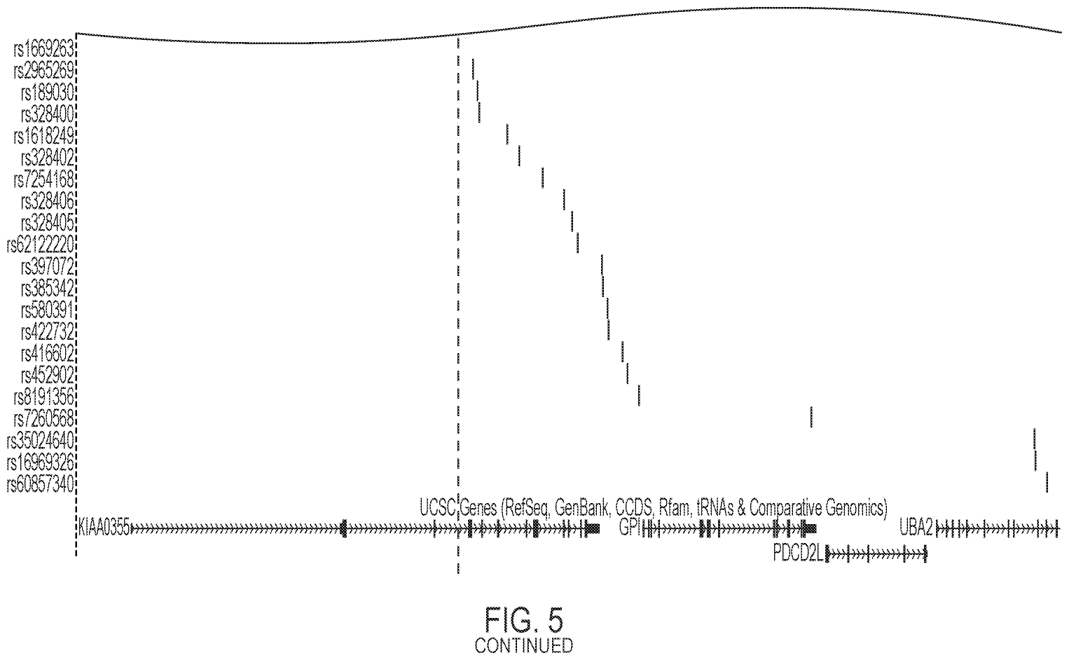

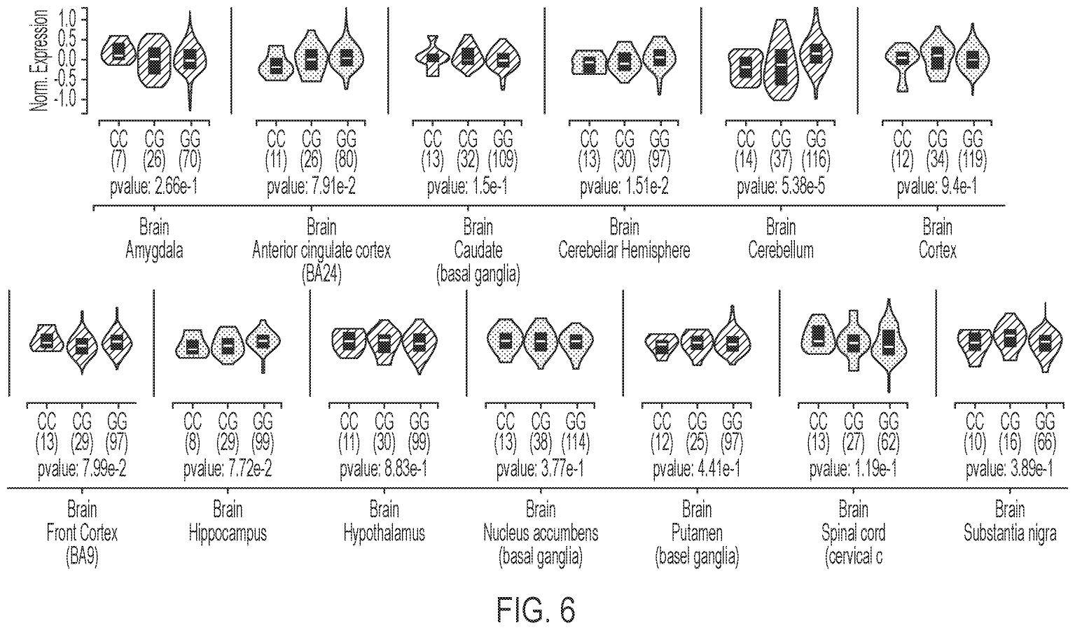

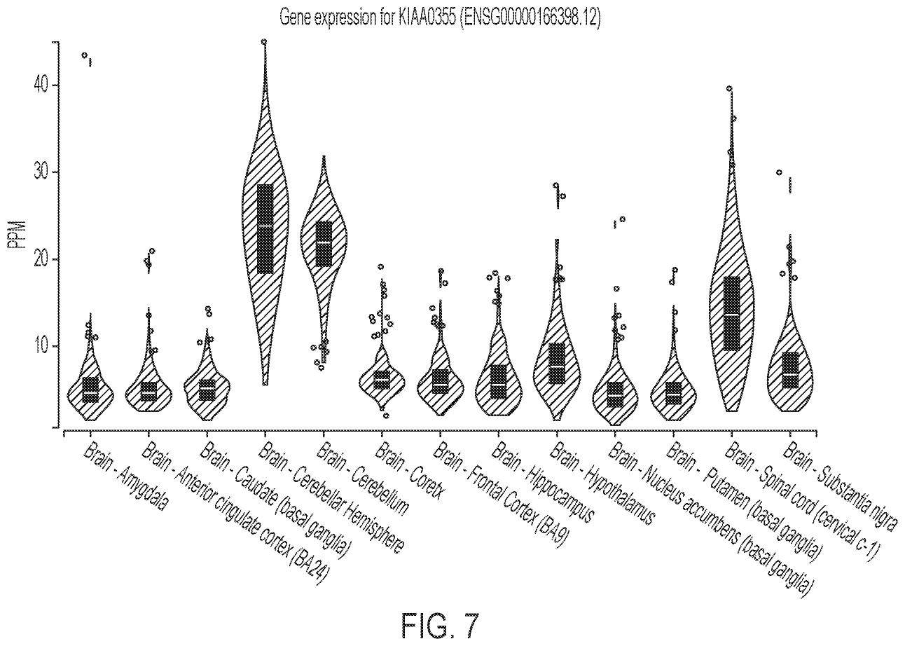

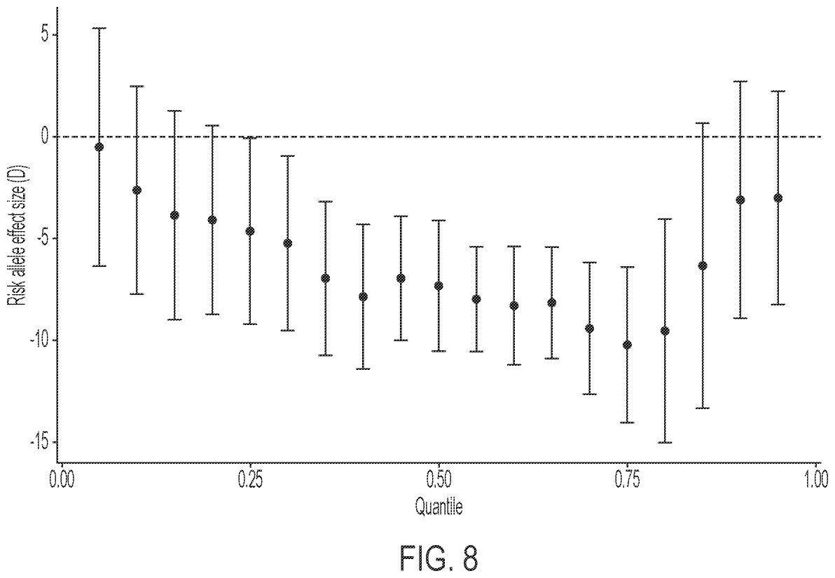

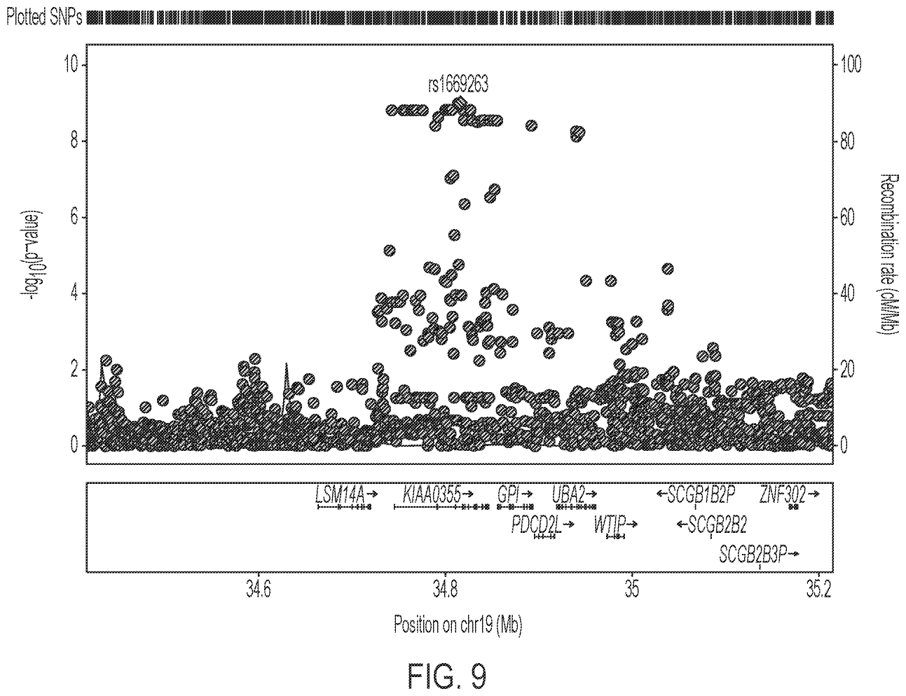

The latent measure utilized in this study, readingT3nocovar, was created based on decoding related tasks. Case and control status was assigned based on performance above or below the mean for readingT3nocovar (FIG. 3).

Results

Using a case-control dichotomization of readingT3nocovar, a peak of SNPs was identified on chromosome 19. With a lambda value of 1.03, these results seem not to be inflated by cryptic ancestry. The peak represents the 39 top SNPs that are associated with performance on the latent measure for reading ability (FIG. 4). These 39 SNPs are described in Table 2, which provides the chromosome, SNP location, the risk allele, and unadjusted p-values from analyzing 359 subjects.

TABLE 2
39 SNPs associated with reading performance.
n = 359 for each of the SNPs below. P-value
is for association with poor reading
performance as determined by the latent
measure for reading ability.

| | SNP | | | |
|---|---|---|---|---|
| | Reference | Base | | |
| | Sequence | Pair | Risk | |
| Chromosome | No. | Location | Allele | P-Value |
| 19 | rs2115487 | 34742162 | A | 1.568e−09 |
| 19 | rs763199675 | 34750567 | G | 3.422e−09 |
| 19 | rs7359931 | 34754797 | G | 1.568e−09 |
| 19 | rs4805079 | 34757224 | A | 1.568e−09 |
| 19 | rs10426700 | 34764563 | T | 1.568e−09 |
| 19 | rs10407101 | 34765113 | T | 1.568e−09 |
| 19 | rs10407640 | 34766588 | G | 1.568e−09 |
| 19 | rs12975032 | 34769256 | C | 1.634e−09 |
| 19 | rs11671239 | 34775721 | A | 1.568e−09 |
| 19 | rs8110966 | 34789302 | C | 4.02e−09 |
| 19 | rs8105306 | 34792333 | T | 2.345e−09 |
| 19 | rs328412 | 34800749 | A | 1.568e−09 |
| 19 | rs328414 | 34802393 | A | 1.568e−09 |
| 19 | rs1664905 | 34805294 | T | 1.568e−09 |
| 19 | rs1669265 | 34805497 | T | 9.527e−08 |
| 19 | rs921476 | 34807200 | A | 1.568e−09 |
| 19 | rs1664904 | 34808389 | T | 8.155e−08 |
| 19 | rs2599553 | 34814089 | A | 1.026e−09 |
| 19 | rs1669263 | 34816031 | C | 1.014e−09 |
| 19 | rs2965269 | 34819331 | A | 1.568e−09 |
| 19 | rs189030 | 34820159 | C | 2.883e−09 |
| 19 | rs328400 | 34820702 | G | 4.727e−07 |
| 19 | rs1618249 | 34826702 | T | 1.568e−09 |
| 19 | rs328402 | 34829261 | C | 2.883e−09 |
| 19 | rs7254168 | 34834364 | A | 3.222e−09 |
| 19 | rs328406 | 34838998 | T | 2.883e−09 |
| 19 | rs328405 | 34840634 | A | 2.883e−09 |
| 19 | rs62122220 | 34841826 | A | 2.883e−09 |
| 19 | rs397072 | 34847014 | A | 2.883e−09 |
| 19 | rs385342 | 34847338 | A | 3.022e−07 |
| 19 | rs580391 | 34848251 | T | 3.022e−07 |
| 19 | rs422732 | 34848450 | C | 2.883e−09 |
| 19 | rs416602 | 34851509 | C | 2.846e−09 |
| 19 | rs452902 | 34852616 | C | 1.832e−07 |
| 19 | rs8191356 | 34855095 | A | 2.846e−09 |
| 19 | rs7260568 | 34892284 | A | 3.901e−09 |
| 19 | rs35024640 | 34940263 | T | 5.557e−09 |
| 19 | rs16969326 | 34940644 | T | 7.5e−09 |
| 19 | rs60857340 | 34943280 | T | 5.557e−09 |

This association was replicated using a separate sample (n=703) of age-matched children drawn from the Genes Reading and Dyslexia (GRaD) study. Of the 39 top SNPs from NHLP, 32 were present in the imputed GRaD dataset. (See Table 3). Covariate data was derived from Truong, et al. (Journal of medical genetics (2019): jmedgenet-2018) and included sex, age at testing, SES, and sufficient principal components to control ancestry. Latent reading scores were calculated for only the subjects that matched. The SRI was swapped for the GORT due to the lack of GORT as part of the GRaD testing battery. Case-control dichotomization and logistic regression were performed as described for the NHLP.

TABLE 3
32 SNPs associated with reading performance as identified in
the GRAD replication studies. n = 703 for each of the SNPs
below. Raw P-values and Benjamini-Hochberg FDR P-values
are reported for all SNPs.

| Chromosome | SNP | P-Value | FDR_BH |
|---|---|---|---|
| 19 | rs328412 | 0.009316 | 0.02084 |
| 19 | rs1664905 | 0.01107 | 0.02084 |
| 19 | rs328414 | 0.01107 | 0.02084 |
| 19 | rs921476 | 0.01107 | 0.02084 |
| 19 | rs8110966 | 0.01157 | 0.02084 |
| 19 | rs8105306 | 0.01157 | 0.02084 |
| 19 | rs1669263 | 0.01368 | 0.02084 |
| 19 | rs2599553 | 0.01368 | 0.02084 |
| 19 | rs11671239 | 0.01415 | 0.02084 |
| 19 | rs416602 | 0.01448 | 0.02084 |
| 19 | rs189030 | 0.01531 | 0.02084 |
| 19 | rs10407101 | 0.0155 | 0.02084 |
| 19 | rs10426700 | 0.0155 | 0.02084 |
| 19 | rs8191356 | 0.01565 | 0.02084 |
| 19 | rs60857340 | 0.0162 | 0.02084 |
| 19 | rs397072 | 0.01627 | 0.02084 |
| 19 | rs4805079 | 0.0164 | 0.02084 |
| 19 | rs12975032 | 0.0167 | 0.02084 |
| 19 | rs7254168 | 0.01674 | 0.02084 |
| 19 | rs328402 | 0.01674 | 0.02084 |
| 19 | rs328406 | 0.01674 | 0.02084 |
| 19 | rs328405 | 0.01674 | 0.02084 |
| 19 | rs10407640 | 0.01705 | 0.02084 |
| 19 | rs1618249 | 0.0173 | 0.02084 |
| 19 | rs35024640 | 0.01742 | 0.02084 |
| 19 | rs2965269 | 0.01815 | 0.02084 |
| 19 | rs422732 | 0.01853 | 0.02084 |
| 19 | rs16969326 | 0.01878 | 0.02084 |
| 19 | rs62122220 | 0.01888 | 0.02084 |
| 19 | rs7359931 | 0.01979 | 0.02111 |
| 19 | rs7260568 | 0.02111 | 0.02179 |
| 19 | rs2115487 | 0.03078 | 0.03078 |

### Conclusion

A new reading and language gene, KIAA0355, has been discovered using SNPs derived from WGS data as part of the New Haven Lexinome Project (NHLP). The results were replicated in an independent, age-matched sample from across the United States of America.

While there have been several published GWAS papers, only two publications demonstrate significant genome-wide association with a genetic marker or gene. These results show strong association with multiple markers from a single gene (KIAA0355) that has never been previously shown to have association with reading or language performance.

### REFERENCES (EXAMPLE 1)

Development NIoCHa (2010) Learning Disabilities (nichd.nih.gov/health/topics/learning_disabilities.cfm).
Huttlin, E. L. et al. Architecture of the human interactome defines protein communities and disease networks. Nature 545, 505-509 (2017).
Lonsdale, J. et al. The Genotype-Tissue Expression (GTEx) project. Nature Genetics 45, 580-585 (2013).
Miller, J. A. et al. Transcriptional landscape of the prenatal human brain. Nature 508, 199-206 (2014).
Pozarickij, A., Williams, C., Hysi, P. G. & Guggenheim, J. A. Quantile regression analysis reveals widespread evidence for gene-environment or gene-gene interactions in myopia development. Communications Biology 2, 1-8 (2019).
Truong, D. T. et al. Multivariate genome-wide association study of rapid automatised naming and rapid alternating stimulus in Hispanic American and African-American youth. Journal of medical genetics (2019).

### Example 2: Genome Wide Association Study in the New Haven Lexinome Project of 415 Subjects

Despite high prevalence and high heritability, few candidate genes have been identified for reading traits. To help address this discrepancy, the New Haven Lexinome Project (NHLP), a longitudinal cohort of students from a typical urban school district in the United States, was analyzed. For the NHLP, genome sequencing, a robust neurobehavioral battery, and neuroimaging were performed. Using logistic regression performed on a mean-split decoding composite variable (N=407), a peak of 31 SNPs on chromosome 19 that achieved the canonical threshold for genome genome-wide significance (rs2599553 P=3.13×10⁻⁸) were identified. Analysis of publicly available expression quantitative trait loci (eQTL) data implicated GARRE1 (also referred to herein as KIAA0355) as a novel candidate gene for decoding performance and suggested a role in cerebellum function. Gene expression data from the Brainspan project further implicated the cerebellum and supported a developmental change. Local ancestry regression implemented through the software package called Tractor, showed that the strongest association for the lead variant was observed in African or Admixed American populations, which are under-represented in reading genetics studies, suggesting one reason why GARRE1 has not previously been associated with a reading phenotype. The chromosome 19 results were replicated in the closely related Genes, Reading, and Dyslexia (GRaD) cohort. a moderating effect of age was also demonstrated, that has implications for the design of future analyses. Finally, the effect of the minor alleles of the lead SNP on reading development through growth curve modeling from Grade 1 through the beginning of Grade 5 were investigated, and showed that children with at least 1 minor allele of rs2599553 persistently underperformed relative to their peers by 0.33 to 0.5 standard deviations on standardized assessments of non-word decoding and reading fluency.

The methods of analysis were performed as described in Example 1 above.

Results

Ancestry Analysis

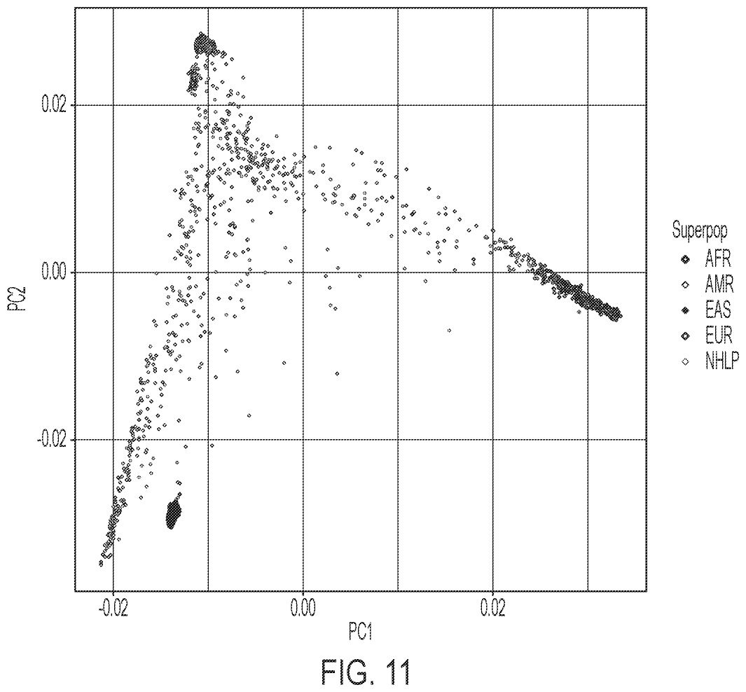

Principal components analysis showed that the sequenced subjects in the NHLP were primarily of global majority race/ethnicities. When plotting PC1 vs. PC2 of the NHLP joined with subjects from the 1000 Genome Project, we observed that our sample overlaps almost completely with subjects from the full AMR or full AFR superpopulations while a small group overlaps with subjects from the full EUR or full EAS superpopulations (FIG. 11). This is consistent with a predominantly Hispanic and African American dataset, supported by self-reported racial category information.

Primary GWAS

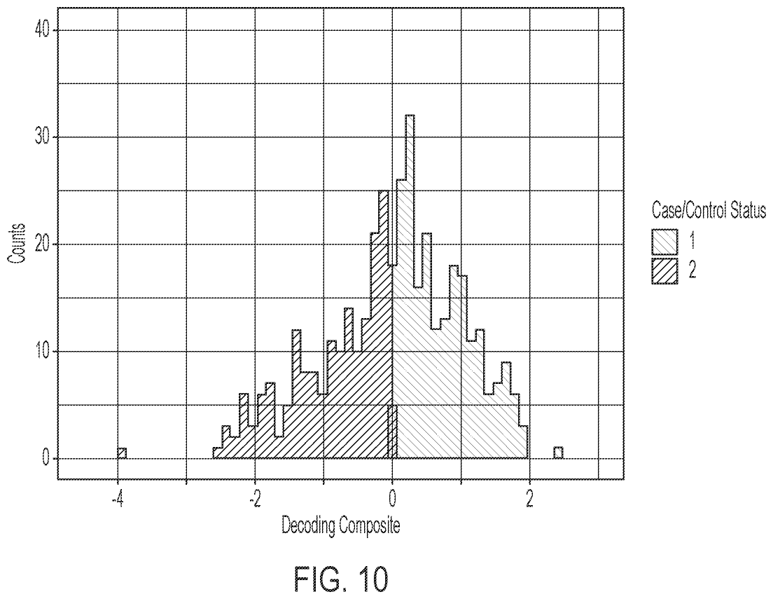

For the primary GWAS analysis in the NHLP, 407 subjects out of an initial 420 subjects with whole genome sequencing data were included. Four subjects were excluded for having self-report Asian ancestry. One subject was excluded for having a sibling in the dataset. Eight subjects lacked sufficient neuropsychological testing data to generate the decoding composite score. No subjects were excluded for a lack of covariate data. Of the remaining 407 subjects, 179 subjects were assigned case (Z-score<0) status and 228 were assigned control (Z-score≥0) status (FIG. 10).

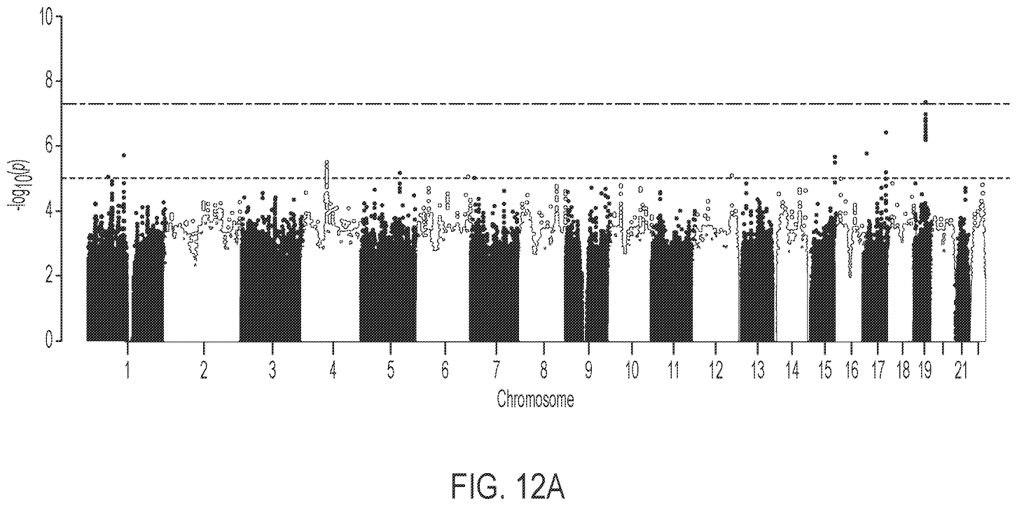

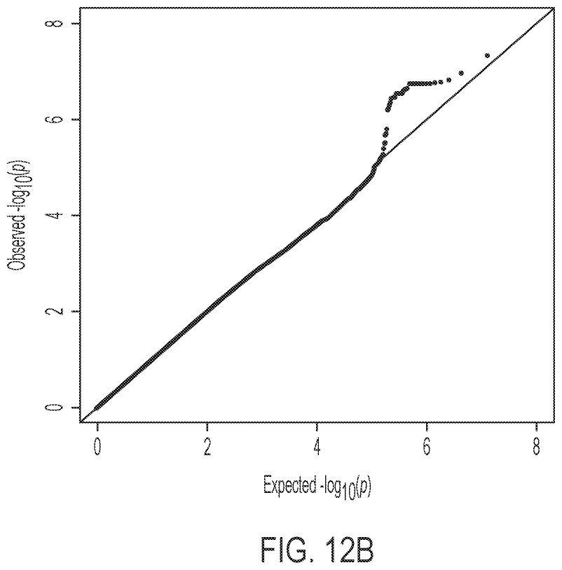

Logistic regression based GWAS of the latent variable derived case/control status identified a cluster of chromosome 19 SNPs centered on and around the gene called GARRE1 (FIG. 12A). Of the SNPs in this cluster, a single SNP (rs2599553), demonstrated a P-value that exceeded the conventional genome-wide significance threshold of 5×10⁻⁸ (FIG. 12A). All 31 genome-wide significant or suggestive SNPs from the primary GWAS are included in Table 7. The lowest observed P-value was 3.13×10⁻⁸ with an odds ratio of 3.141 for rs2599553 (Table 7). Manual inspection of the Quantile-Quantile (Q-Q) plot (FIG. 12B), and the calculated lambda test statistic for inflation of 1.04149, both supported the assertion that the primary GWAS was well-controlled for confounding due to admixture.

Post Hoc Analysis of Rs2599553 and the Decoding Composite

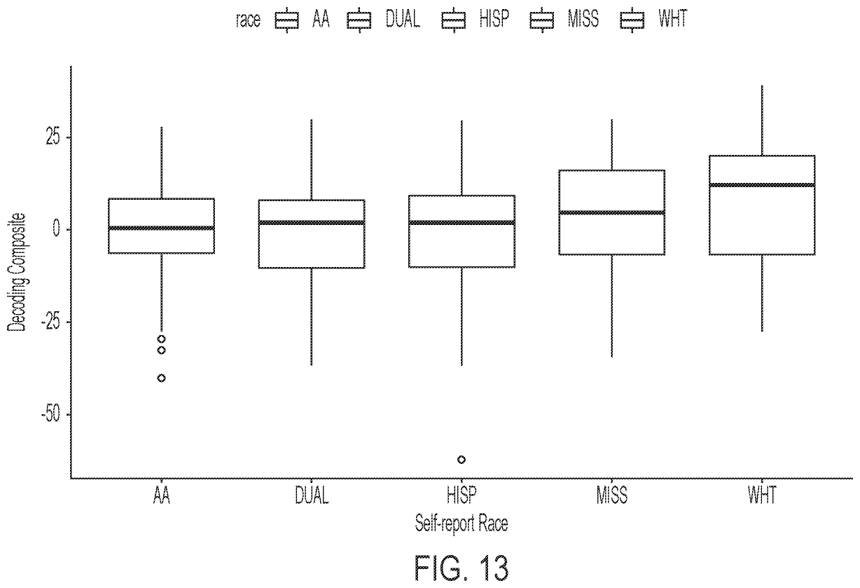

To investigate the most common causes of P-value inflation in GWAS studies, differences in allele frequency between populations and differences in phenotype frequency between populations, we performed a series of post-hoc statistical tests on our top SNP from the GWAS and our raw decoding composite (Tables 8 and 9). To test whether or not there is a significant difference in allele counts between self-reported racial categories in the NHLP, we performed a 5×2 Chi-squared test (Table 8). This showed a test statistic of 1.0391 with a corresponding P-value of 0.90381 and did not support a significant deviation in allele counts between self-reported racial groupings [χ² (4, 407)=1.0391, p=n.s.]. To test for the possibility of a difference in the distribution of decoding composite scores between self-report racial categories, we performed a one-way ANOVA (Table 9) and Tukey's HSD test. Box-plots of raw decoding composite scores were plotted by self-report racial category in FIG. 13. The ANOVA showed an F-test statistic of 2.07 and a corresponding P-value of 0.084, indicating no statistically significant differences in decoding score by self-report racial groupings (F(4,402)=2.07, p=0.084; Table 8). Tukey's HSD tests for differences in means between each pair of self-report racial categories, and is generally applied as a follow-up to ANOVA if results are significant. Despite no significant association between self-ID racial category and performance on the decoding composite, we performed Tukey's HSD. No significant effects were observed for any pair of self-reported racial categories, suggesting that confounding is unlikely (data not shown).

Tractor Analysis

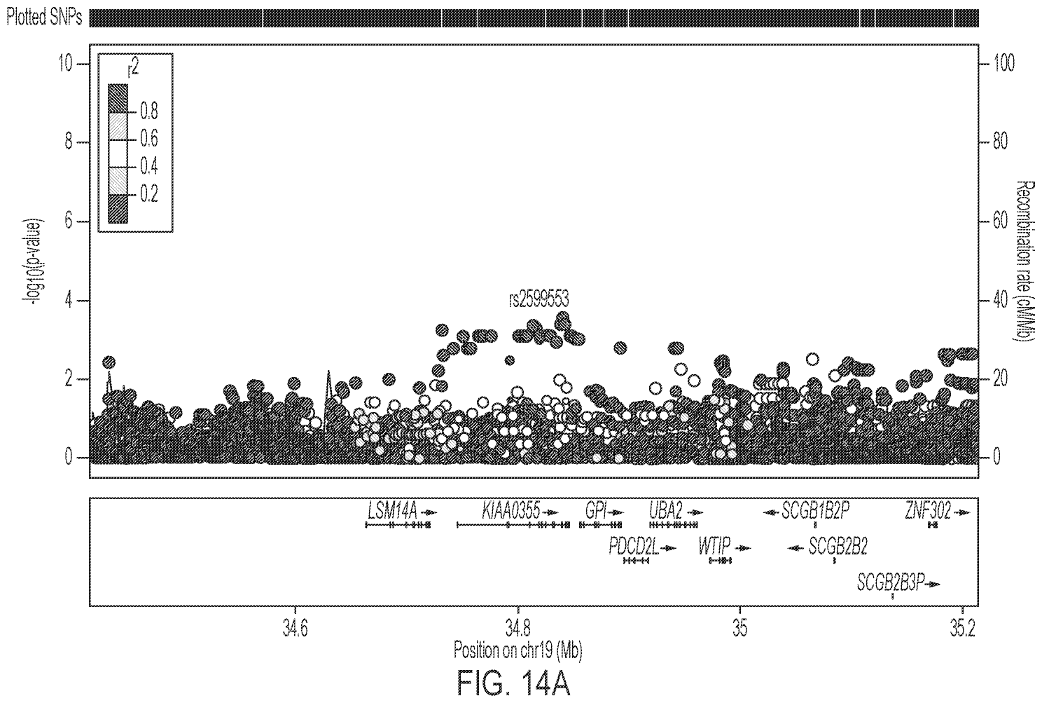

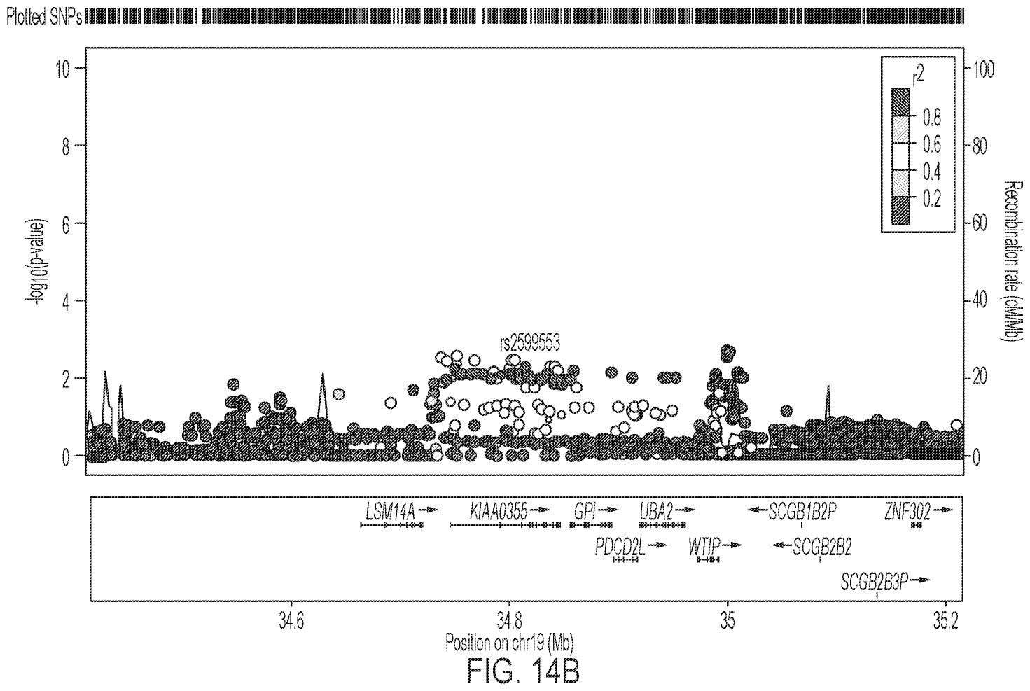

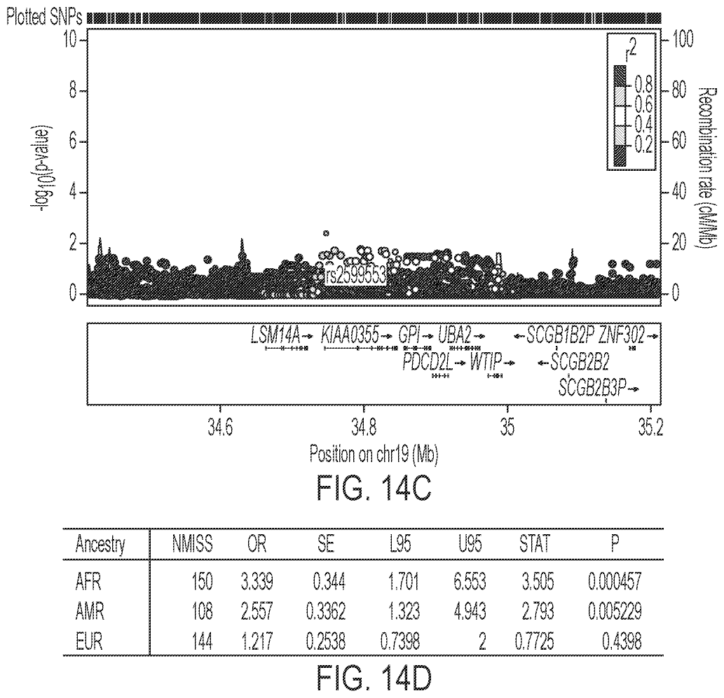

Tractor was used to partition the phased, joint-called NHLP genotype files into three separate VCF files corresponding to the African, European, and Admixed American (AA) ancestry-specific haplotype tracts. Individual GWAS for each ancestry of the N=415 individuals were performed using the covariates described above. Individual ancestry GWAS indicated that the segment of interest on chromosome 19 most strongly associated with decoding performance in standard GWAS bore the strongest signal in AFR ancestry (rs2599553; P=0.000457, OR=3.339; FIG. 14A). For comparison, AMR ancestry also showed an association of P=0.005229, and OR=2.557 (FIG. 14B) while EUR ancestry only showed a nonsignificant P-value of 0.4398, and OR=1.217 (FIG. 14C). These differences in P-values suggest that the signal for this genetic variant in the primary GWAS mostly came from the African ancestry present in the admixed sample and highlight the utility of accounting for local ancestry in admixed cohorts.

Bioinformatic Analysis

The 31 SNPs comprising the chromosome 19 peak span 250 Kbp and four non-overlapping genes: GARRE1, GPI, PDCDL2, and UBA2. All 31 SNPs are non-coding, and 22 overlap GARRE1. LD analysis showed that all 31 SNPs had R 2 values above 0.95, indicating a single locus, and could not be used to differentiate between the four genes. Thirty SNPs were observed in the GTEx eQTL dataset, and all were eQTLs for GARRE1 expression in the cerebellum. The lack of eQTL evidence for any other of the genes in the chromosome 19 peak strongly implicates GARRE1 as a candidate gene for decoding performance. Bulk mRNA sequencing from the GTEx project showed peak brain expression in the cerebellum with a median TPM value for GARRE1 of 23.71 in cerebellar hemisphere and 21.86 in cerebellum (cerebellar hemisphere and cerebellum are treated as replicates sampled at two separate times by two separate teams), supporting the eQTL observations.

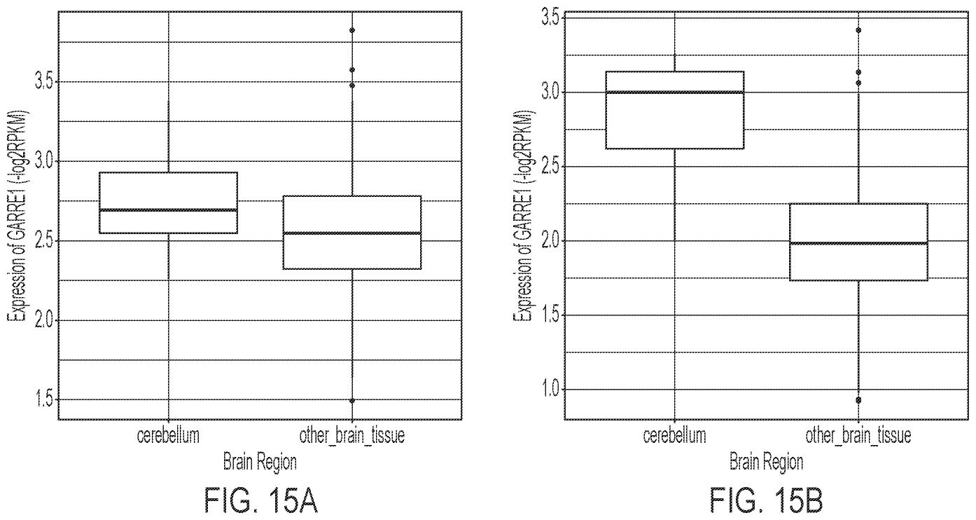

BrainSpan data showed that in human fetal samples between 12 to 37 weeks post-conception (FIG. 15A), GARRE1 expression in the cortex and cerebellum are equivalent (Wilcoxon's Rank Sum test p=0.12; N=11 subjects). Beginning at 4 months postnatal age and extending well into adulthood there is significantly higher expression of GARRE1 in the cerebellum relative to all other brain tissues (Wilcoxon's Rank Sum test p=6.88×10⁻¹¹; N=21 subjects) (FIG. 15B).

Data from the gnomAD project suggested that GARRE1 is intolerant to loss of function (pLOF) mutations with a pLI score of 0.97 and a ratio of observed to expected pLOF mutations of 0.17. The expectation under a neutral model is that 46.4 pLOF mutations would be observed in a dataset the size of gnomAD, however, only 8 were observed for GARRE1. In contrast, slightly fewer missense mutations were observed than expected with 507 observed against an expected 622. The numbers of synonymous mutations fell within the expected ratio, with 272 observed against an expected 254.6. Together these data indicate that GARRE1 is performing an important function in humans that requires two intact genes for successful reproduction.

Replication in GRaD

For replication, we chose the GRaD Study because subjects were assessed with a robust battery that included single-word decoding skills, they were previously genotyped with a large number of SNPs, and because the GRaD sample has a broad representation of Hispanic-American and African-American children from different regions of the U.S. Using the same set of covariates from the primary GWAS in the NHLP and an analogous mean split decoding composite, we achieved p<0.05 for all SNPs in the locus (N=632; Table 10). The pairwise R 2 values were above 0.95 for all 31 SNPs, suggesting that there is only a single effective test, avoiding the need for a multiple testing correction. The best performing SNP in the NHLP, rs2599553, had a P-value of 0.015 in GRad; the best results in GRaD were from rs2965269, P-value=0.012. Interestingly, we were only able to replicate when we age-matched GRaD subjects to NHLP (restricting the GRaD to 7-10 year old subjects), before mean splitting the composite, suggesting a possible gene-by-environment (GxE) effect.

Moderation Analysis

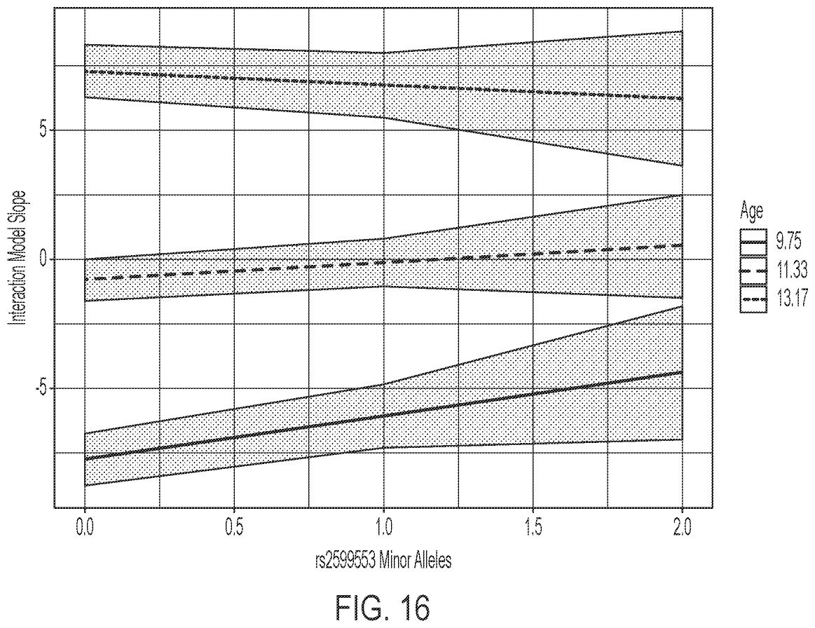

To investigate a potential age-based GxE effect, we performed a SNPxAGE moderation analysis for the lead SNP (rs2599553) from the primary analysis in NHLP. Using the quantitative, normally distributed, decoding composite for the full GRaD cohort (N=1,291), we performed a regression with and without a rs2599553×Age interaction term. Both models included age, sex, a binary SES variable, and ten PCs to control for admixture. The main effect of rs2599553 genotype was significant only when the interaction term was included in the model (P<0.05); the P-value for rs2599553×Age was also less than 0.05 (Table 11). These analyses indicate that age had a significant moderating influence on the effect of rs2599553 on decoding performance. Stratifying age by quantile, we observed that the youngest subjects performed worse on decoding than the oldest tranche of subjects. However, the direction of effect was different between the youngest and oldest subjects. Subjects in the bottom 25% of the age distribution showed a positive direction of effect with increasing numbers of the minor allele of rs2599553. Subjects in the top 25% of the age distribution showed a negative direction of effect with increasing numbers of the minor allele of rs2599553. The central 50% of the age/performance distribution curve was relatively flat (FIG. 16).

Relative Risk

The top SNP from the primary GWAS, rs2599553, was coded according to a dominance model and used to calculate the relative risk of case status. Of the 323 subjects in this analysis, 101 were coded as having risk due to minor alleles of rs2599553, and of those, 39.6% (n=40) were RD cases. 222 subjects were coded as having no allele risk, and of those, 21.2% (n=47) were RD cases. Taken together, having the minor allele of rs2599553 conferred a 2.11 relative risk for meeting RD criteria at the start of Grade 2, assuming a conservative prevalence of 11% for reading disability in the general population. (Fletcher et al., 2007). Expressed differently, the top SNP from the primary GWAS conferred a 111% elevated risk of meeting the criteria for RD in Grade 2.

Growth Curve Analysis

Subjects from the NHLP were tested on a nationally normed reading assessment, the WJ-III, a maximum of nine times from the start of Grade 1 until the fall of Grade 5. Among the 412 children who completed at least one assessment point and had available genetic data, longitudinal data density was as follows: Grade 1 start, n=383; Grade 1 end, n=343; Grade 2 start, n=368; Grade 2 end, n=340; Grade 3 start, n=380; Grade 3 end, n=361; Grade 4 start, n=359; Grade 4 end, n=191; Grade 5 start, n=174. Median number of assessments per child was seven, ranging from one to nine assessments. No differences across GARRE1 risk categories were observed for longitudinal data density (X²(8)=2.00, p=0.90) or number of assessments (X²(8)=5.09, p=0.75).

The following WJ-III subtests were used to formulate growth curves: Letter-Word Identification, measuring single-word identification; Word Attack, measuring orthographically-regular non-word decoding; Passage Comprehension, measuring reading of connected text for meaning via a doze procedure; Reading Fluency, measuring both fluency and comprehension of connected text. In the analysis that followed modeling developmental trajectories over time, both raw scores and standardized scores were used. In the first case, raw score models addressed absolute skill growth over time. In the second case, standard score models provided a picture representing how children change relative to the normative developmental expectations as characterized by the normative sample of the test. Standard scores on the WJ-III have a mean of 100, and a standard deviation of 15. In the standard score outcome analyses, a standard score of 100 was within developmental expectations; a standard score of 85 was one standard deviation below developmental expectations. A standard score below 85 is often used to indicate significant problems acquiring reading skill and as one of the criteria for diagnosing a reading-specific learning difficulty. A standard score of 90 is often used as a clinical cut-off representing ‘average’ reading ability.

Growth curve models were formulated following best practices. (Hox et al., 2010; Snijders and Bosker, 2011) PROC MIXED in SAS/STAT software version 9.04 of the SAS System for Linux, was used to fit all multilevel growth models. After data screening and assessment of basic assumptions for distribution and outliers, the shape of individual growth trajectories was investigated empirically prior to analysis, using visual inspection of each child's trajectory from the fall of Grade 1 to the fall of Grade 5. Competitive approaches using different models of growth were then evaluated (e.g., linear versus higher-order versus growth to an asymptote, etc.). The most parsimonious and well-fitting growth model included an intercept centered at the Grade 1 start, with a linear growth component. Models of raw score test performance required an additional quadratic function to represent a general deceleration of growth rates over the observational period. Since the nine measurement timepoints have educational significance (i.e., beginning and end of each school year), but specific measurement dates varied per child, a hybrid model for time was implemented. Several models for time were considered against each other with a two-component model providing the best fit to the repeated measures elements in the model. In the first component, the nine fixed measurement occasions were modeled as random effects. In the second component, the number of days between measurements for each child was used to model the within-subject residual variance, using a spatial power covariance matrix. (Macchiavelli and Moser, 1997)

The following covariates were entered into the model as fixed-effect predictors of intercept, growth, and deceleration in the case of raw score models: biological sex, low versus average SES defined by parental report of having received some form of social assistance, and ten principal components to control for ancestry. In no case did a covariate predict growth or deceleration, therefore they were both pruned from all growth models. The top SNP from the primary GWAS, rs2599553, was recoded for a dominance model and incorporated as follows: as a fixed-effect predictor of skill level differences across the study span; as a predictor of individual growth and change; and as a predictor of growth deceleration in the case of raw score models.

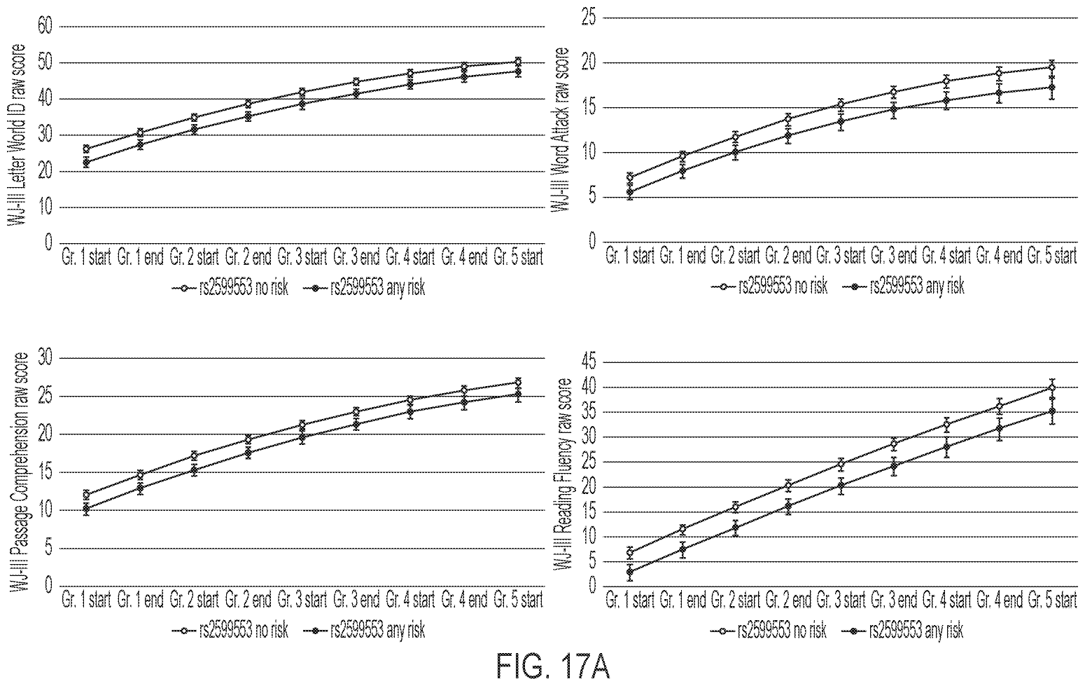

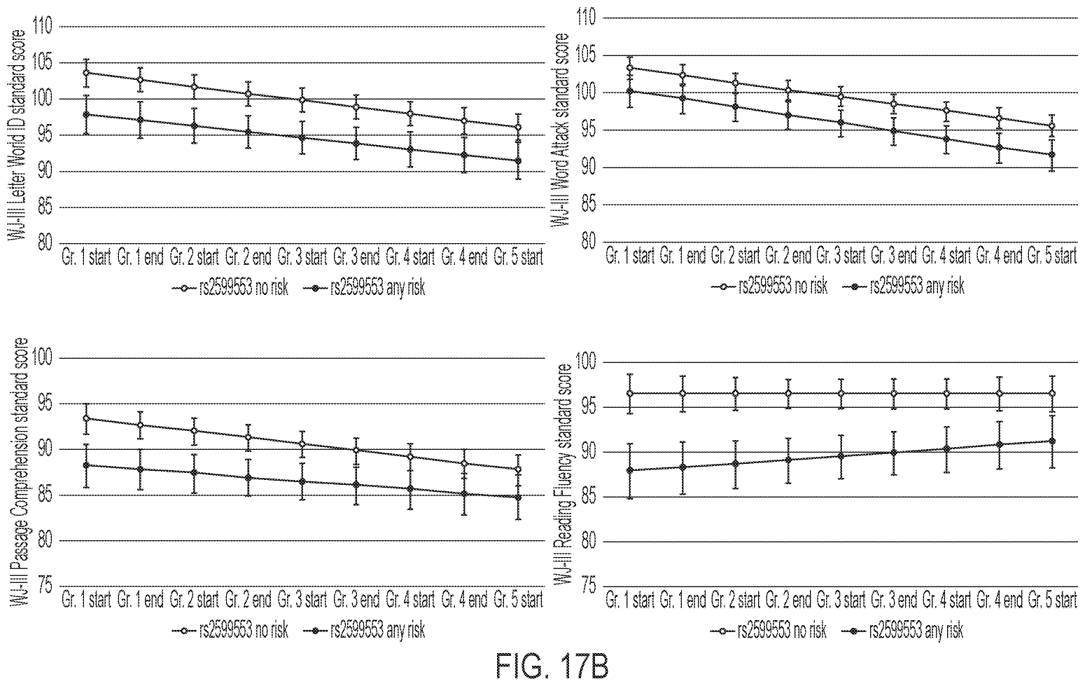

Across all four raw score growth models, substantial child-to-child variability was observed in the random effects for intercept, growth rates, and deceleration, indicating that growth over time was not influenced by the timing of when a child was enrolled, in Grade 1 or 2. As depicted in FIGS. 17A-17B, the general pattern of growth in reading skill was an increasing raw score ability that decelerated over the study period. Also depicted in FIG. 17A-17B is consistently suppressed reading skill for those with the GARRE1 risk allele compared to those without. This effect was replicated across all four reading measures and across all observation points from Grade 1 to Grade 5. Table 12 details the difference in reading scores between risk and no risk, along with standard errors and confidence intervals of this difference.

Standard score growth models portray a different picture, placing each child's score in relation to developmental expectations. In the standard score growth models, risk group was a significant predictor of ability for all dimensions of reading: Letter Word Identification, Word Attack, Passage Comprehension, and Reading Fluency. These differences in risk group reading performance were maintained at all time points. Table 13 provides a test of the estimated mean difference between risk groups at each timepoint per standard score outcome measures. Given that the standard deviation of the standard scores is 15, Table 13 indicates that the developmental risk conferred by GARRE1 ranges from one-third (Word Attack outcome) to over one-half of a standard deviation (Reading Fluency outcome) below age expectations for reading performance.

### Discussion

Utilizing a mean-split transformation of a latent phenotype indexing decoding in poor performing students from the longitudinal NHLP, we identified an association between GARRE1 on chromosome 19 and decoding performance. Association results exceeded genome-wide significance thresholds, and were well-controlled for ancestry, sex, and SES. We replicated our finding in an age-matched subset of the GRaD cohort. In addition, we observed that age moderates the effect of rs2599553 on decoding performance with opposing directions of effect for different quantiles of age.

Further analysis with Tractor allowed for the partitioning of a single VCF from admixed subjects into three separate, ancestry-specific files containing alleles from chromosome segments inherited from a single ancestry. This allowed for fine-scale control of population structure in admixed and mixed cohorts, detection of ancestry-specific differences in allele frequencies and effect sizes, and identification of the ancestral source of our primary GWAS signal. eQTL data from the GTEx Project suggested that the GWAS signal likely originated from GARRE1, which may play a role in cerebellar development and function. Expression data from Brainspan suggested that there is a developmental change in the expression of GARRE1, from equal expression in cortex and cerebellum to predominantly cerebellum, which persists through adulthood.

In the NHLP, gene risk group significantly predicted reading skills, as measured by four related dimensions of reading at the beginning of Grade 1. The risk effect was present at all testing points to the beginning of Grade 5 for all measures. These results indicate that children with the minor allele of rs2599553 begin Grade 1 with lower reading ability and that this gap is maintained throughout the primary school years until the beginning of Grade 5. There was no relationship between GARRE1 risk and the rate of acquisition of reading skills.

When the outcome was children's performance relative to developmental expectations, as represented by the normative sample of the reading test, risk associated with GARRE1 was also demonstrated. As illustrated by the growth curve analyses (FIG. 8), these differences in reading performance were maintained at every time point until the end of Grade 5, though there was no relationship between GARRE1 risk and change in clinical severity over time. Of particular clinical significance, children with one rs2599553 minor allele performed below average and showed the greatest deficits in passage comprehension and reading fluency relative to their peers at the start of Grade 1. The relative deficits in passage comprehension increased in clinical significance over time. By the end of Grade 5, children with the minor allele as a group approached the clinical cutoff for a specific learning deficit in reading.

GARRE1, RAC1, and the Cerebellum

Little is known about the function of GARRE1 (previously known as KIAA0355). In 2018, a proximity mapping experiment localized GARRE1 protein to cytoplasmic granules in HEK293 cells, suggesting a role in mRNA processing and protein expression. Later optogenetic studies showed a physical interaction between GARRE1 and RAC1. RAC1 is a relatively well characterized Rho GTPase associated with a diverse collection of cellular processes, including lamellipodia formation. Lamellipodia are transient cell structures associated with cellular migration, including neurons, that have been reported to play a role in reading and language problems. RAC1 mutations have been associated with severe developmental disorders, including at least one case report associated with cerebellar hypoplasia and microcephaly. (Reijnders et al., 2017)

While somewhat controversial, previous reports have described difficulties with balance and keeping time—frequently associated with cerebellar function—linked to RD. The cerebellum plays a significant role in skill automatization for a wide variety of tasks, including reading. The cerebellar deficit hypothesis of dyslexia suggests that there is a cerebro-cerebellar link, and deficits in cerebellar development impair automatization of reading skills, leading to lifelong difficulty in developing the fluent reading skills required for success in school and some employment opportunities. The Simple View of reading development suggests that children undergo a developmental change from focusing on decoding performance to reading comprehension in early elementary education (Hoover and Gough, 1990). In successful readers, decoding becomes more automatized and is less emphasized as children learn to read. This developmental change generally occurs around Grade 3 in the US (8 to 9 years of age), consistent with the developmental window in the NHLP subjects from this study and the matched segment of the GRaD used for replication. Taken together, our results lend genetic support to the cerebellar deficit hypothesis. Variation in GARRE1 may lead to modulation of RAC1 activity or expression that presents as subtle changes in the cerebellum which lead to difficulty in automatizing word decoding. Further studies are needed to support or reject this model.

Reading, Genetics, and Gene-by-Environment Effects

In addition to supporting to the cerebellar deficit hypothesis, these results suggest a note of caution for nascent efforts to meta-analyze multiple reading and language samples together. For these efforts to be successful, care must be taken to account for possible confounding through gene-by-environment effects. Children pass through several developmental windows as they become fluent readers and as brain circuits mature. Hypothetically, if more reading-related traits show a similar interaction effect pattern as observed in the GRaD sample, with a positive direction of effect at one age and negative at another, a meta-analysis could lead to a null result as different directions of effect in different subgroups of the sample essentially cancel each other out. Careful study design will be critical in studies going forward to maximize the potential from often underpowered and heterogeneous samples common to the genetics of reading.

This study highlights the importance of wide and deep phenotyping, longitudinal study design, and inclusion of diverse populations for genetic studies. We demonstrate a viable path for novel genetic discovery and candidate gene identification, even in small primary samples, through the construction of a holistic approach that integrates GWAS, replication, and bioinformatics. Our results add further evidence in support of genetic screening to presymptomatically identify children who are at significant risk for reading deficits. They also suggest that future analyses of the NHLP could show new correlations between genetic variants and variable responses to a comprehensive intervention, a potential clinically useful tool for counseling students and their parents and for modifying curricula.

### REFERENCES

Atkinson E (2020) eatkinson/Tractor. Available at: github.com/eatkinson/Tractor [Accessed Mar. 2, 2020].
Atkinson E G, Maihofer A X, Kanai M, Martin A R, Karczewski K J, Santoro M L, Ulirsch J C, Kamatani Y, Okada Y, Finucane H K, Koenen K C, Nievergelt C M, Daly M J, Neale B M (2020) Tractor: A framework allowing for improved inclusion of admixed individuals in large-scale association studies. bioRxiv:2020.05.17.100727.
Dalvie S, Koen N, Duncan L, Abbo C, Akena D, Atwoli L, Chiliza B, Donald K A, Kinyanda E, Lochner C, Mall S, Nakasujja N, Newton C R, Ramesar R, Sibeko G, Teferra S, Stein D J, Koenen K C (2015) Large Scale Genetic Research on Neuropsychiatric Disorders in African Populations is Needed. EBioMedicine 2:1259-1261.
DePristo M A, Banks E, Poplin R, Garimella K V, Maguire J R, Hartl C, Philippakis A A, del Angel G, Rivas M A, Hanna M, McKenna A, Fennell T J, Kernytsky A M, Sivachenko A Y, Cibulskis K, Gabriel S B, Altshuler D, Daly M J (2011) A framework for variation discovery and genotyping using next-generation DNA sequencing data. Nat Genet 43:491-498.
Gibbs R A et al. (2003) The International HapMap Project. Nature 426:789-796.
Hoover W A, Gough P B (1990) The simple view of reading. Read Writ 2:127-160.
Hox J J, Moerbeek M, van de Schoot R (2010) Multilevel Analysis: Techniques and Applications, 2nd ed. New York, NY: Routledge.
Li H, Durbin R (2009) Fast and accurate short read alignment with Burrows-Wheeler transform. Bioinformatics 25:1754-1760.
Li H, Handsaker B, Wysoker A, Fennell T, Ruan J, Homer N, Marth G, Abecasis G, Durbin R, Subgroup 1000 Genome Project Data Processing (2009) The Sequence Alignment/Map format and SAMtools. Bioinformatics 25:2078-2079.
Loh P-R, Danecek P, Palamara P F, Fuchsberger C, Reshef Y A, Finucane H K, Schoenherr S, Forer L, McCarthy S, Abecasis G R, Durbin R, Price A L (2016) Reference-based phasing using the Haplotype Reference Consortium panel. Nature Genetics 48:1443-1448.
Lonsdale J et al. (2013) The Genotype-Tissue Expression (GTEx) project. Nature Genetics 45:580-585.
Lovett M W, Frijters J C, Wolf M, Steinbach K A, Sevcik R A, Morris R D (2017) Early intervention for children at risk for reading disabilities: The impact of grade at intervention and individual differences on intervention outcomes. Journal of Educational Psychology 109:889-914.
Lyon G R, And Others (1997) Progress and Promise in Research in Learning Disabilities. Learning Disabilities: A Multidisciplinary Journal 8:1-6.
Macchiavelli R E, Moser E B (1997) Analysis of Repeated Measurements with Ante-Dependence Covariance Models. Biometrical Journal 39:339-350.
Machiela M J, Chanock S J (2015) LDlink: a web-based application for exploring population-specific haplotype structure and linking correlated alleles of possible functional variants. Bioinformatics 31:3555-3557.
Manichaikul A, Mychaleckyj J C, Rich S S, Daly K, Sale M, Chen W-M (2010) Robust relationship inference in genome-wide association studies. Bioinformatics 26:2867-2873.
Maples B K, Gravel S, Kenny E E, Bustamante C D (2013) RFMix: A Discriminative Modeling Approach for Rapid and Robust Local-Ancestry Inference. The American Journal of Human Genetics 93:278-288.
Martin E R, Tunc I, Liu Z, Slifer S H, Beecham A H, Beecham G W (2018) Properties of global- and local-ancestry adjustments in genetic association tests in admixed populations. Genetic Epidemiology 42:214-229.
McKenna A, Hanna M, Banks E, Sivachenko A, Cibulskis K, Kernytsky A, Garimella K, Altshuler D, Gabriel S, Daly M, DePristo M A (2010) The Genome Analysis Toolkit: a MapReduce framework for analyzing next-generation DNA sequencing data. Genome Research 20:1297-1303.
Miller J A et al. (2014) Transcriptional landscape of the prenatal human brain. Nature 508:199-206.
Mills R E, Pittard W S, Mullaney J M, Farooq U, Creasy T H, Mahurkar A A, Kemeza D M, Strassler D S, Ponting C P, Webber C, Devine S E (2011) Natural genetic variation caused by small insertions and deletions in the human genome. Genome Res Available at: genome.cshlp.org/content/early/2011/04/25/gr.115907.110 [Accessed Nov. 6, 2019].
Peterson R L, Pennington B F (2015) Developmental Dyslexia. Annu Rev Clin Psychol 11:283-307.
Price A L, Patterson N J, Plenge R M, Weinblatt M E, Shadick N A, Reich D (2006) Principal components analysis corrects for stratification in genome-wide association studies. Nat Genet 38:904-909.
Price A L, Weale M E, Patterson N, Myers S R, Need A C, Shianna K V, Ge D, Rotter J I, Tones E, Taylor K D, Goldstein D B, Reich D (2008) Long-Range L D Can Confound Genome Scans in Admixed Populations. Am J Hum Genet 83:132-135.
Purcell S, Neale B, Todd-Brown K, Thomas L, Ferreira M A, Bender D, Maller J, Sklar P, de Bakker P I, Daly M J, Sham P C (2007) PLINK: a tool set for whole-genome association and population-based linkage analyses. Am J Hum Genet 81:559-575.
R Core Team (2016) R: A language and environement for statistical computing. Vienna, Austria: R Foundation for Statistical Computing. Available at: r-project.org.
Reijnders M R F et al. (2017) RAC1 Missense Mutations in Developmental Disorders with Diverse Phenotypes. The American Journal of Human Genetics 101:466-477.
Schatschneider C, Torgesen J K (2004) Using our current understanding of dyslexia to support early identification and intervention. Journal of Child Neurology 19:759-765.
Sherry S T, Ward M, Sirotkin K (1999) dbSNP-database for single nucleotide polymorphisms and other classes of minor genetic variation. Genome Res 9:677-679.
Sirugo G, Williams S M, Tishkoff S A (2019) The Missing Diversity in Human Genetic Studies. Cell 177:26-31.
Snijders T A B, Bosker R (2011) Multilevel analysis. An introduction to basic and advanced multilevel modeling, 2nd edition (1st edition 1999). London: SAGE Publications Ltd.
The Genomes Project Consortium (2015) A global reference for human genetic variation. Nature 526:68-74.
Truong D T, Adams A K, Paniagua S, Frijters J C, Boada R, Hill D E, Lovett M W, Mahone E M, Willcutt E G, Wolf M, Defries J C, Gialluisi A, Francks C, Fisher S E, Olson R K, Pennington B F, Smith S D, Bosson-Heenan J, Gruen J R (2019) Multivariate genome-wide association study of rapid automatised naming and rapid alternating stimulus in Hispanic American and African-American youth. Journal of medical genetics.
Van der Auwera G A, Carneiro M O, Hartl C, Poplin R, del Angel G, Levy-Moonshine A, Jordan T, Shakir K, Roazen D, Thibault J, Banks E, Garimella K V, Altshuler D, Gabriel S, DePristo M A (2013) From FastQ data to high confidence variant calls: the Genome Analysis Toolkit best practices pipeline. Curr Protoc Bioinformatics 43:11.10.1-33.
Wanzek J, Vaughn S, Scammacca N K, Metz K, Murray C S, Roberts G, Danielson L (2013) Extensive Reading Interventions for Students With Reading Difficulties After Grade 3. Review of Educational Research 83:163-195.
Wanzek J, Wexler J, Vaughn S, Ciullo S (2010) Reading interventions for struggling readers in the upper elementary grades: a synthesis of 20 years of research. Read Writ 23:889-912.

Tables for Example 2.

TABLE 4
NHLP GWAS sample demographics (N = 407 w/all covariate data).

| | |
|---|---|
| Sex (M:F) | 192:215 |
| Age (Years; mean) | 6.0-10.25 (7.42) |
| SES (High:Low) | 43:364 |
| Mean-split Decoding Composite (Below | 179:228 |
| mean:Above mean) | |
| Self-Report European Ancestry (N subj.) | 19 |
| Self-Report African Ancestry (N subj.) | 95 |
| Self-Report Hispanic Ancestry (N subj. ) | 107 |
| Self-Report Dual Ancestry (N subj.) | 99 |
| Failed to Report Ancestry (N subj.) | 96 |

TABLE 5
Measures performed in the NHLP. Measures included in the decoding composite
phenotype are in bold.

| Measure | Subtests | Targeted Construct(s) | Author |
|---|---|---|---|
| Test of Word Reading Efficiency, | Sight Word Efficiency | Reading accuracy and fluency | Torgesen. Wagner, & |
| 2nd Ed. (TOWRE-2) | Phonetic Decoding | for whole words and nonwords | Rashotte, 2012 |
| | Efficiency | | |
| Woodcock-Johnson III Tests of | Letter-Word ID | Academic achievement in a | Woodcock, McGrew. & |
| Achievement (WJ III ACH) | Reading Fluency | variety of reading and math | Mather, 2001 |
| | Passage Comprehension | tasks targeting specific | |
| | Word Attack | cognitive abilities | |
| | Calculation | | |
| | Math Fluency | | |
| | Applied Problems | | |
| Gray Oral Reading Tests, 5th Ed. | Reading Fluency | Oral reading ability including | Wiederhott & Bryant, |
| (GORT-5) | Reading Comprehension | rate, accuracy, fluency and | 2012 |
| | | comprehension | |

TABLE 6
Primary GWAS results for chromosome 19 sorted by base
pair position.

| rsID | BP | Minor Allele | OR | P-Value |
|---|---|---|---|---|
| rs2115487 | 34742162 | A | 3.184 | 1.52E−07 |
| rs11084762 | 34750567 | G | 2.988 | 1.88E−07 |
| rs7359931 | 34754797 | G | 3.247 | 9.22E−08 |
| rs4805079 | 34757224 | A | 3.247 | 9.22E−08 |
| rs10426700 | 34764563 | T | 3.247 | 9.22E−08 |
| rs10407101 | 34765113 | T | 3.247 | 9.22E−08 |
| rs10407640 | 34766588 | G | 3.247 | 9.22E−08 |
| rs12975032 | 34769256 | C | 3.217 | 1.19E−07 |
| rs11671239 | 34775721 | A | 3.247 | 9.22E−08 |
| rs328412 | 34800749 | A | 3.247 | 9.22E−08 |
| rs328414 | 34802393 | A | 3.278 | 7.49E−08 |
| rs1664905 | 34805294 | T | 3.247 | 9.22E−08 |
| rs921476 | 34807200 | A | 3.247 | 9.22E−08 |
| rs2599553 | 34814089 | A | 3.381 | 3.13E−08 |
| rs1669263 | 34816031 | C | 3.316 | 5.47E−08 |
| rs2965269 | 34819331 | A | 3.257 | 1.27E−07 |
| rs189030 | 34820159 | C | 3.165 | 1.48E−07 |
| rs1618249 | 34826702 | T | 3.214 | 1.20E−07 |
| rs328402 | 34829261 | C | 3.165 | 1.48E−07 |
| rs7254168 | 34834364 | A | 3.272 | 9.29E−08 |
| rs328406 | 34838998 | T | 3.165 | 1.48E−07 |
| rs328405 | 34840634 | A | 3.212 | 1.23E−07 |
| rs62122220 | 34841826 | A | 3.165 | 1.48E−07 |
| rs397072 | 34847014 | A | 3.165 | 1.48E−07 |
| rs422732 | 34848450 | C | 3.165 | 1.48E−07 |
| rs416602 | 34851509 | C | 3.167 | 1.70E−07 |
| rs8191356 | 34855095 | A | 3.167 | 1.70E−07 |
| rs7260568 | 34892284 | A | 3.203 | 1.58E−07 |
| rs35024640 | 34940263 | T | 3.186 | 1.34E−07 |
| rs16969326 | 34940644 | T | 3.135 | 2.00E−07 |
| rs60857340 | 34943280 | T | 3.186 | 1.34E−07 |

TABLE 7
Chi-Squared test for difference of minor/major allele counts across self-report

| rs2599553 | AA (N = 95) | HISP (N = 107) | WHT (N = 19) | DUAL (N = 99) | NA (N = 96) | |
|---|---|---|---|---|---|---|
| Minor | 30 | 41 | 7 | 38 | 36 | Test Statistic = 1.0391 |
| Major | 160 | 173 | 31 | 160 | 156 | P = 0.90381 |
| MAF | 0.158 | 0.192 | 0.184 | 0.192 | 0.1875 | |

TABLE 8
One-way ANOVA results for differences in mean across
self-report racial groupings

| | DF | Sum Sq | Mean Sq | F-Test Value | P-Value |
|---|---|---|---|---|---|
| Self-Report Race | 4 | 2014 | 503.5 | 2.07 | 0.084 |
| Residuals | 402 | 97809 | 243.5 | | |

TABLE 9
GRAD Candidate SNP replication

| SNP | Number of Subjects | P |
|---|---|---|
| rs2599553 | 632 | 0.01501 |
| rs2965269 | 633 | 0.01243 |

TABLE 10
GRaD Moderation analysis results.

| | Dependent variable: | | | |
|---|---|---|---|---|
| | GRAD Latent Reading Variable | | | |
| | No Interation | | Age/SNP Interaction | |
| Constant | −47.464*** | (−51.394, −43.534) | −50.308*** | (−54.952, −45.665) |
| rs2699653_A | 0.603 | (−0.578, 1,783) | 7.988* | (1.434, 14,541) |
| age | 4.148*** | (3.832, 4.463) | 4.398*** | (4.015, 4.781) |
| SEX | 2.164** | (0.850, 3.477) | 2.207** | (0.895, 3.519) |
| SES_CC | −2.817*** | (−4.020, −1.213) | −2.694*** | (−4.097, −1.291) |
| PC1 | 37.469** | (13.421, 61.517) | 37.505** | (13.495, 61.516) |
| PC2 | 61.280*** | (36.189, 86.370) | 61.142*** | (36.091, 86.193) |
| PC3 | −10.597 | (−35.025, 13.831) | −11.148 | (−35.542, 13.246) |
| PC4 | 5.522 | (−18.165, 29.209) | 5.088 | (−18.564, 28.740) |
| PC5 | 4.959 | (−18.652, 28.570) | 4.100 | (−19.486, 27.685) |
| PC6 | −3.449 | (−27.094, 20.196) | −3.518 | (−27.125, 20.090) |
| PC7 | −3.085 | (−26.767, 20.597) | −2.285 | (−25.940, 21.370) |
| PC8 | 21.818 | (−1.737, 45.372) | 23.119 | (−0.425, 46.664) |
| PC9 | −11.444 | (−35.211, 12.324) | −11.489 | (−35.219, 12.241) |
| PC10 | 19.076 | (−4.507, 42.658) | 18.708 | (−4.839, 42.256) |
| rs2599553_A:age | | | −0.647* | (−1.212, −0.082) |
| Observations | 1,291 | | 1,291 | |
| R² | 0.402 | | 0.405 | |
| Adjusted R² | 0.396 | | 0.398 | |
| Residual Std. Error | 11.962 | (df = 1276) | 11.943 | (df = 1275) |
| F Statistic | 61.315*** | (df = 14; 1276) | 57.745** | (df = 15; 1275) |

Note:
*p < 0.05;
**p < 0.01;
***p < 0.001

TABLE 11
Woodcock-Johnson III Raw Score Mean Differences

| Subscale | Timepoint | Estimate | SE | P-value | Upper 95% Cl | Lower 95% Cl |
|---|---|---|---|---|---|---|
| Letter Word ID | Grade 1 Start | 3.6 | 0.93 | 0.0001 | 1.8 | 5.5 |
| | Grade 1 End | 3.5 | 0.88 | <.0001 | 1.8 | 5.3 |
| | Grade 2 Start | 3.4 | 0.65 | <.0001 | 1.8 | 5.1 |
| | Grade 2 End | 3.3 | 0.84 | <.0001 | 1.7 | 5.0 |
| | Grade 3 Start | 3.2 | 0.84 | 0.0001 | 1.6 | 4.9 |
| | Grade 3 End | 3.1 | 0.85 | 0.0003 | 1.5 | 4.8 |
| | Grade 4 Start | 3.0 | 0.87 | 0.0006 | 1.3 | 4.7 |
| | Grade 4 End | 2.9 | 0.91 | 0.0015 | 1.1 | 4.7 |
| | Grade 5 Start | 2.8 | 0.96 | 0.0036 | 0.93 | 4.7 |
| Word Attack | Grade 1 Start | 1.6 | 0.51 | 0.0021 | 0.57 | 2.6 |
| | Grade 1 End | 1.7 | 0.49 | 0.0009 | 0.69 | 2.6 |
| | Grade 2 Start | 1.7 | 0.49 | 0.0005 | 0.77 | 2.7 |
| | Grade 2 End | 1.8 | 0.51 | 0.0004 | 0.83 | 2.8 |
| | Grade 3 Start | 1.9 | 0.53 | 0.0004 | 0.86 | 3.0 |
| | Grade 3 End | 2.0 | 0.57 | 0.0006 | 0.86 | 3.1 |
| | Grade 4 Start | 2.4 | 0.62 | 0.0009 | 0.85 | 3.3 |
| | Grade 4 End | 2.2 | 0.68 | 0.0016 | 0.83 | 3.5 |
| | Grade 5 Start | 2.2 | 0.74 | 0.0026 | 0.79 | 3.7 |
| [symbol] ssage Comprehension | Grade 1 Start | 1.9 | 0.51 | 0.0002 | 0.91 | 2.9 |
| | Grade 1 End | 1.9 | 0.48 | 0.0001 | 0.91 | 2.8 |
| | Grade 2 Start | 1.8 | 0.47 | 0.0001 | 0.89 | 2.7 |
| | Grade 2 End | 1.8 | 0.46 | 0.0002 | 0.84 | 2.7 |
| | Grade 3 Start | 1.7 | 0.47 | 0.0003 | 0.77 | 2.6 |
| | Grade 3 End | 1.6 | 0.49 | 0.0008 | 0.68 | 2.6 |
| | Grade 4 Start | 1.6 | 0.52 | 0.0023 | 0.57 | 2.6 |
| | Grade 4 End | 1.5 | 0.55 | 0.006 | 0.45 | 2.6 |
| | Grade 5 Start | 1.5 | 0.60 | 0.014 | 0.31 | 2.7 |
| Reading Fluency | Grade 1 Start | 4.0 | 0.98 | <.0001 | 2.1 | 5.9 |
| | Grade 1 End | 4.1 | 0.95 | <.0001 | 2.2 | 6.0 |
| | Grade 2 Start | 4.2 | 0.96 | <.0001 | 2.3 | 6.0 |
| | Grade 2 End | 4.2 | 1.00 | <.0001 | 2.3 | 6.2 |
| | Grade 3 Start | 4.3 | 1.07 | <.0001 | 2.2 | 6.4 |
| | Grade 3 End | 4.4 | 1.17 | 0.0002 | 2.1 | 6.7 |
| | Grade 4 Start | 4.5 | 1.28 | 0.0005 | 2.0 | 7.0 |
| | Grade 4 End | 4.6 | 1.42 | 0.0014 | 1.8 | 7.4 |
| | Grade 5 Start | 4.7 | 1.56 | 0.0031 | 1.6 | 7.7 |

[symbol]

indicates data missing or illegible when filed

TABLE 12
Woodcock-Johnson III Standard Score Mean Differences

| Subscale | Timepoint | Estimate | SE | P-value | Upper 95% Cl | Lower 95% Cl |
|---|---|---|---|---|---|---|
| Letter Word ID | Grade 1 Start | 5.7 | 1.7 | 0.0007 | 2.4 | 9.0 |
| | Grade 1 End | 5.6 | 1.6 | 0.0005 | 2.4 | 8.7 |
| | Grade 2 Start | 5.4 | 1.5 | 0.0004 | 2.4 | 8.4 |
| | Grade 2 End | 5.3 | 1.5 | 0.0004 | 2.4 | 8.2 |
| | Grade 3 Start | 5.2 | 1.5 | 0.0005 | 2.3 | 8.1 |
| | Grade 3 End | 5.0 | 1.5 | 0.0007 | 2.1 | 7.9 |
| | Grade 4 Start | 4.9 | 1.5 | 0.0013 | 1.9 | 7.9 |
| | Grade 4 End | 4.8 | 1.6 | 0.0024 | 1.7 | 7.8 |
| | Grade 5 Start | 4.6 | 1.6 | 0.0047 | 1.4 | 7.8 |
| Word Attack | Grade 1 Start | 3.1 | 1.3 | 0.022 | 0.45 | 5.7 |
| | Grade 1 End | 3.2 | 1.3 | 0.012 | 0.71 | 5.6 |
| | Grade 2 Start | 3.3 | 1.2 | 0.0062 | 0.94 | 5.6 |
| | Grade 2 End | 3.4 | 1.2 | 0.0035 | 1.1 | 5.7 |
| | Grade 3 Start | 3.5 | 1.1 | 0.0022 | 1.3 | 5.8 |
| | Grade 3 End | 3.6 | 1.2 | 0.0018 | 1.4 | 5.9 |
| | Grade 4 Start | 3.8 | 1.2 | 0.0017 | 1.4 | 6.1 |
| | Grade 4 End | 3.9 | 1.2 | 0.0019 | 1.4 | 6.3 |
| | Grade 5 Start | 4.0 | 1.3 | 0.0025 | 1.4 | 6.6 |
| Passage Comprehension | Grade 1 Start | 5.2 | 1.5 | 0.0005 | 2.3 | 8.0 |
| | Grade 1 End | 4.9 | 1.4 | 0.0004 | 2.2 | 7.6 |
| | Grade 2 Start | 4.6 | 1.3 | 0.0005 | 2.1 | 7.2 |
| | Grade 2 End | 4.4 | 1.3 | 0.0007 | 1.9 | 6.9 |
| | Grade 3 Start | 4.1 | 1.3 | 0.0013 | 1.6 | 6.6 |
| | Grade 3 End | 3.8 | 1.3 | 0.0032 | 1.3 | 6.4 |
| | Grade 4 Start | 3.6 | 1.3 | 0.0085 | 0.91 | 6.2 |
| | Grade 4 End | 3.3 | 1.4 | 0.022 | 0.49 | 6.1 |
| | Grade 5 Start | 3.0 | 1.5 | 0.049 | 0.012 | 6.0 |
| Reading Fluency | Grade 1 Start | 8.7 | 2.0 | <.0001 | 4.8 | 12.6 |
| | Grade 1 End | 8.3 | 1.8 | <.0001 | 4.7 | 11.8 |
| | Grade 2 Start | 7.8 | 1.6 | <.0001 | 4.6 | 11.0 |
| | Grade 2 End | 7.4 | 1.5 | <.0001 | 4.4 | 10.4 |
| | Grade 3 Start | 7.0 | 1.5 | <.0001 | 4.1 | 9.9 |
| | Grade 3 End | 6.5 | 1.5 | <.0001 | 3.6 | 9.4 |
| | Grade 4 Start | 6.1 | 1.5 | <.0001 | 3.1 | 9.1 |
| | Grade 4 End | 5.7 | 1.6 | 0.0006 | 2.4 | 8.9 |
| | Grade 5 Start | 5.2 | 1.8 | 0.0036 | 1.7 | 8.8 |

TABLE 13
Random Effects Covariance Parameter Estimates for Woodcock-Johnson III Raw
Scores

| | Letter Word ID | | | Word Attack | | | Passage Comprehension | | | Reading Fluency | | |
|---|---|---|---|---|---|---|---|---|---|---|---|---|
| Parameter | Estimate | SE | P-value | Estimate | SE | P-value | Estimate | SE | P-value | Estimate | SE | P-value |
| Intercept | 66 | 5.5 | <.0001 | 14 | 1.6 | <.0001 | 18 | 1.7 | <.0001 | 61 | 7.1 | <.0001 |
| Slope | 3.6 | 0.56 | <.0001 | 1.6 | 0.32 | <.0001 | 1.4 | 0.24 | <.0001 | 7.0 | 1.4 | <.0001 |
| Deceleration | 0.026 | 0.0066 | <.0001 | 0.032 | 0.0042 | 0.0022 | 0.012 | 0.0032 | <.0001 | 0.068 | 0.019 | <.0001 |
| Within-subject | 0 49 | 0.10 | <.0001 | 0.60 | 0.059 | <.0001 | 0.58 | 0.075 | <.0001 | 0.64 | 0.059 | <.0001 |
| covariance | | | | | | | | | | | | |

TABLE 14
Random Effects Covariance Parameter Estimates for Woodcock-Johnson III
Standard Scores

| | Letter Word 10 | | | Word Attack | | | Passage Comprehension | | | Reading Fluency | | |
|---|---|---|---|---|---|---|---|---|---|---|---|---|
| Parameter | Estimate | SE | P-value | Estimate | SE | P-value | Estimate | SE | P-value | Estimate | SE | P-value |
| Intercept | 232 | 19 | <.0001 | 154 | 14 | <.0001 | 189 | 17 | <.0001 | 1121 | 97 | <.0001 |
| Slope | 6.2 | 1.3 | <.0001 | 7.5 | 1.5 | <.0001 | 13 | 2.0 | <.0001 | 175 | 17 | <.0001 |
| Deceleration | 0.019 | 0.014 | 0.68 | 0.048 | 0.019 | 0.0058 | 0.096 | 0.023 | <.0001 | 1.5 | 0.17 | <.0001 |
| Within-subject | 0.58 | 0.070 | <.0001 | 0.51 | 0.19 | <.0001 | 0.68 | 0.044 | <.0001 | 0.86 | 0.014 | <.0001 |
| covariance | | | | | | | | | | | | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. (canceled)

2. A method of detecting one or more single nucleotide polymorphisms (SNPs) in human chromosome 19 in a sample, wherein the SNPs have any one of the reference sequence (rs) numbers listed in Table 2 or any one of the reference sequence (rs) numbers listed in Table 3, or any one of the SNPs listed in Table 6, or any one of the rs numbers listed in Table 6,
wherein the identity of the SNPs determines the risk of poor reading performance in an individual, and
wherein the sample is obtained from an individual and comprises nucleic acid.

3. The method of claim 2, wherein the presence of a minor allele at any one of the SNPs indicates the presence or predisposition for poor reading performance.

4. A method of assessing the risk of low reading performance or the presence of or predisposition for low reading performance in an individual, the method comprising:
detecting, in a sample obtained from an individual, the identity of at least one single nucleotide polymorphism (SNP) having a reference sequence (rs) number listed in Table 2 or a reference sequence number listed in Table 3, or a SNP listed in Table 6, or an rs number listed in Table 6,
wherein the nucleotide identity of the at least one SNP is the corresponding risk allele according to Table 2 or Table 6,
wherein the sample comprises nucleic acid.

5. (canceled)

6. The method of claim 4, wherein
the is in the KIAA0355 (GARRE1) gene on chromosome 19 (19q13.11), and
wherein the identity of the SNP is associated with a latent measure of reading ability.

7. (canceled)

8. The method of claim 4, wherein the SNP is a non-coding SNP.

9. The method of claim 6, wherein the latent measure of reading ability is decoding ability.

10. The method of claim 6, wherein the SNP is located within base pair locations (BP) 34,348,356-34,359,412, and wherein the presence of a minor allele at any one of the rs numbers indicates the presence or predisposition for poor reading ability.

11.-12. (canceled)

13. The method of claim 2, wherein the SNP has a reference sequence number of rs1669263 and a nucleotide identity of C.

14. The method of claim 2, wherein the SNP has a reference sequence number of rs2599553 and a nucleotide identity of A.

15. The method of claim 2, wherein the reading performance is measured by at least one of: letter word identification, word attack, passage comprehension, and reading fluency.

16. The method of claim 2, wherein the detecting comprises nucleic acid sequencing techniques.

17. The method of claim 2, wherein the detecting comprises using next generation sequencing or microarray genotyping.

18. The method of claim 2, wherein the sample is a tissue sample, a cell sample, a saliva sample, a blood sample, or a urine sample.

19. (canceled)

20. The method of claim 2, wherein the SNP is in KIAA0355 (GARRE1), GPI, PDCD2L, or UBA2.

21. (canceled)

22. The method of claim 2, wherein if the individual has any one of the risk alleles in Table 2 or Table 3 or Table 6, the method further comprises monitoring the individual from whom the sample was obtained to assess whether development of a learning or reading disability occurs and if development occurs, treating the individual for the learning or reading disability, wherein treating comprises providing interventions, including services and materials, including but not limited to: using special teaching techniques; making classroom modifications, such as providing extra time to complete tasks and taped tests to permit the individual to hear, rather than read the tests; using books on tape; using word-processing programs with spell-check features; helping the individual learn through multisensory experiences; teaching coping tools; and providing services to strengthen the individual's ability to recognize and pronounce words.

23. The method of claim 2, wherein if the individual has any one of the risk alleles in Table 2 or Table 6, the method further comprises administering an intelligence quotient (IQ) test.

24. A method of analyzing human chromosome 19 (19q13.11) by detecting in a sample, obtained from a human and comprising nucleic acid, at least one non-coding single nucleotide polymorphism (SNP) having a reference sequence (rs) number in Table 2 or a reference sequence (rs) number in Table 3, or a SNP listed in Table 6, or an rs number listed in Table 6, comprising:
(a) combining the sample with polynucleotides that hybridize, under highly stringent conditions, with the at least one non-coding SNP when the nucleotide identity of the at least one-non SNP corresponds is the corresponding risk allele in Table 2 or Table 6; and
(b) determining whether hybridization of the polynucleotides in (a) occurs, wherein the occurrence of hybridization the polynucleotides indicates that the human has the risk allele and is susceptible to or has developed a reading disability.

25. The method of claim 4, wherein the SNP has a reference sequence number of rs1669263 and a nucleotide identity of C.

26. The method of claim 4, wherein the SNP has a reference sequence number of rs2599553 and a nucleotide identity of A.

27. The method of claim 18, wherein the tissue sample or cell sample is obtained by swabbing, scraping, swiping, biopsying, or a combination thereof.
